(19) United States
(12) Patent Application Publication
(10) Pub. No.: US 2026/0256431 A1
(43) Pub. Date: Sep. 3, 2026

(54) IMAGING OF MATRIX METALLOPROTEINASE INHIBITORS

(71) Applicants: Micro Vide, LLC, Columbia, SC (US); Yale University, New Haven, CT (US)

(72) Inventors: Albert J. Sinusas, Guiford, CT (US); Francis G. Spinale, Mill Spring, NC (US); Paul Heerdt, Greenwich, CT (US); Stephanie Thorn, New Haven, CT (US)

(73) Assignees: Micro Vide, LLC, Columbia, SC (US); Yale University, New Haven, CT (US)

(21) Appl. No.: 18/873,120

(22) PCT Filed: Jun. 9, 2023
(86) PCT No.: PCT/US2023/025007

§ 371 Date: Dec. 9, 2024

Related U.S. Application Data
(60) Provisional application No. 63/351,063, filed on Jun. 10, 2022.

(51) Int. Cl. A61B 6/03; A61B 6/00; A61B 6/50; A61K 51/04

(52) U.S. Cl. CPC A61B 6/032; A61B 6/037; A61B 6/4417; A61B 6/481; A61B 6/503; A61B 6/5205; A61K 51/041; A61K 51/0497

(57) ABSTRACT

The present disclosure is directed in part to methods of imaging a subject's lung, including but not limited to the purpose of evaluating a pulmonary infection in a subject. The method includes administering an imaging agent comprising a ^{99m}Tc-labeled matrix metalloproteinase targeted radiotracer to a subject, acquiring an image of the subject's lung using SPECT/CT imaging, and measuring an amount of the imaging agent bound to matrix metalloproteinase in the subject's lung (or localized to the lung). Additionally, the present disclosure is directed in part to a method of high resolution imaging of a subject's heart tissue.

### RELATED APPLICATIONS

This application claims the benefit of the filing date of U.S. Provisional Application No. 63/351,063, filed on Jun. 10, 2022, the entire contents of which are incorporated by reference herein.

### FEDERAL RESEARCH STATEMENT

This invention was made with government support under Grant Nos. 1R42HL099258, 1R42HL131280, R01HL137365 and R01HL159459 awarded by the National Institutes of Health. The government has certain rights in the invention.

### BACKGROUND

Acute respiratory distress syndrome (ARDS) is a serious pulmonary condition. Activation of matrix metalloproteinases (MMPs) have been implicated in the progression of pulmonary infections and other pulmonary conditions, such as ARDS. MMPs are zinc-containing enzymes that are capable of degrading extracellular matrix proteins. MMP proteolytic activity and extracellular matrix degradation is dependent on the comparative balance between MMPs and tissue inhibitor of metalloproteinases (TIMPs). Radiolabeled metalloproteinase inhibitors have primarily been developed for tumor imaging and for cardiovascular applications, which focused on quantification of relative myocardial ^{99m}Tc-RP805 uptake or myocardial uptake as a percentage of injected dose of radiotracer from static images. ARDS is characterized by persistent pulmonary neutrophilic inflammation, edema, and pulmonary hemorrhage, which complicates quantitative analysis of SPECT imaging of the lungs with molecularly targeted radiotracers using traditional techniques. However, quantification and ultimately modulation of MMP activity may improve outcome in patients afflicted with a pulmonary infection.

As such, there is a need to improve methods for imaging and quantifying regional and global tissue MMP activity in a subject suspected of being afflicted with a pulmonary infection.

### SUMMARY

Certain aspects of the present disclosure are directed to a method of imaging a subject's lung. Methods disclosed herein include administering an imaging agent comprising a ^{99m}Tc-labeled matrix metalloproteinase targeted radiotracer to a subject's lung, acquiring an image of the subject's lung using hybrid digital single-photon emission computed tomography (SPECT)/computed tomographic (CT) imaging, and measuring an amount of the imaging agent bound to matrix metalloproteinase in the subject's lung. Imaging agents may be administered to the lung of a subject via systemic administration such as intravenous administration.

Other aspects of the present disclosure are directed to a method of evaluating a pulmonary infection in a subject. The method includes administering an imaging agent comprising a ^{99m}Tc-labeled matrix metalloproteinase targeted radiotracer to a subject's lung; acquiring an image of the subject's lung using SPECT/CT imaging; and measuring an amount of the imaging agent bound to matrix metalloproteinase in the subject's lung.

The presence of the imaging agent bound to a matrix metalloproteinase in an amount higher than an amount in a reference subject indicates the subject is afflicted with a pulmonary infection.

Various embodiments apply to the foregoing aspects. For example, acquiring an image of the subject's lung may comprise acquiring dynamic SPECT images for kinetic modeling. The kinetic modeling may comprise a two tissue compartment model having four parameters. The kinetic modeling may comprise an exponential model having two equilibrating components and one trapping component. Alternatively or additionally, acquiring dynamic SPECT images may have an imaging time of about 1 hour.

Acquiring an image and/or kinetic modeling may further comprise determining a regional air fraction V_{A} and/or determining a fractional blood volume V_{B}.

The foregoing methods may further comprise normalizing the amount of the imaging agent bound to matrix metalloproteinase in the subject's lung to the subject's weight and an administered amount of the imaging agent. The methods may further comprise evaluating tissue-capillary integrity and tracer exchange rates.

The methods may further comprise segmenting one or more lobes of the subject's lung, optionally according to one or more ranges of Hounsfield density of a CT scan.

Still other aspects of the present disclosure are directed to a method of high resolution imaging of a subject's heart tissue. The method includes administering to the subject's heart tissue a first imaging agent comprising a ^{99m}Tc-labeled matrix metalloproteinase targeted radiotracer; acquiring an image of the subject's heart tissue using SPECT/CT imaging; and quantifying an amount of the first imaging agent bound to a matrix metalloproteinase in the subject's heart tissue.

In some embodiments, the method further comprises administering to the subject's heart tissue a second imaging agent substantially concurrently with the first imaging agent. The second imaging agent may comprise or be labelled with I-123, In-111, xenon-133 or gallium-67. Typically, gamma energy of the first imaging agent and of the second imaging agent are separable and thus discernable from each other. As an example, the first imaging agent may have a gamma energy of about 140 kV and the second imaging agent may have a gamma energy of about 70 kV.

In some embodiments, the first imaging agent may be used to detect, monitor and/or measure MMP presence or level, MMP activity level, or extracellular matrix degradation and the second imaging agent may be used to detect, monitor and/or measure perfusion such as myocardial perfusion. In related embodiments, acquiring images from the first and second imaging agents is done substantially simultaneously.

In some embodiments, administering to the subject's heart tissue a first imaging agent comprises administering, for example systemically, a dose of the first imaging agent within a range from about 15 mCi to about 30 mCi and wherein administering to the subject's heart tissue a second imaging agent comprises administering, for example systemically, a dose of the second imaging agent within a range from about 2.0 mCi to about 3.5 mCi.

These and other features and aspects, embodiments and advantages of the present invention will become better understood with reference to the following description and appended claims.

### BRIEF DESCRIPTION OF THE DRAWINGS

A full and enabling disclosure of the present disclosure is set forth more particularly in the remainder of the specification, including reference to the accompanying figures, in which:

FIG. 1A depicts reconstructed gated blood pool image derived from the first 5 min following the administration of ^{99m}Tc-RP805.

FIG. 1B depicts a left ventricle volume curve derived from the data in FIG. 1A to determine end-diastolic and end-systolic volumes and left ventricular ejection fraction.

FIG. 1C depicts a comparison of the LVEF obtained in animals (n=5) 3 days post-MI derived from the ^{99m}Tc-RP805 gated blood pool analysis versus the gold standard of the gated contrast CT.

FIG. 1D depicts a Bland-Altman plot demonstrating the excellent correlation for the LVEF of the analyzed gated contrast cardiac CT image versus the ^{99m}Tc-RP805 gated blood pool.

FIG. 1E depicts a time activity curve for the myocardium (Global LV Myocardium
top plot) and left ventricle cavity blood pool (bottom plot).

FIG. 2 depicts ^{99m}Tc-RP805 uptake in a 3-day post-ischemia reperfusion swine at 1 and 4 hr post injection.

FIG. 3 depicts a 17-segment polar map display of the uptake of ^{99m}Tc-RP805 in the infarcted antero-septal zone.

FIGS. 4A-F depict quantitative indices of lung, color coded radiotracer activity distribution superimposed on chest CT images. FIG. 4A: Summed PET (KBq/ml); FIG. 4B: Computed Tomography; FIG. 4C: SUV (g/ml); FIG. 4D: Ratio (ml/cm³); FIG. 4E: Ki_{SAIF} (ml/cm³/min); FIG. 4F: Ki_{Parlak} (ml/cm³/min).

FIG. 5 depicts semi-automated lobar segmentation of lungs using a 3D slicer.

FIG. 6 depicts fusion of CT derived lobar volumes of interest (VOIs) with masked ^{99m}Tc-RP805 SPECT images for SPECT quantification.

FIG. 7 depicts a quantification of dynamic ^{99m}Tc-RP805 SPECT images acquired in pig following lavage of a left lung over 2 hours post radiotracer injection for each lobar segment (left) and left and right lungs (right panel, left lung is upper plot and right lung is lower plot).

FIG. 8 depicts segmentation and quantification of ^{99m}Tc-RP805 SPECT/CT images based on ranges of Hounsfield density. Green is <−600HU, yellow is from −600 to −300 HU, and red is >−300 HU.

FIG. 9 depicts quantification of lung uptake of ^{99m}Tc-RP805 based on segmentation of a lung into three categories based on CT density (HU). Low intensity CT range (<−600 HU, lower plot), intermediate intensity (from −600 to −300 HU, middle plot), and high intensity (>−300 HU, top plot).

FIG. 10 depicts a proposed analysis of radiotracer activity in a lung. Top left panel shows air, blood, tissue for control and air, blood, and tissue and water for ARDS. (See Chen et al. J. Nucl. Med. 2017 58(2):201-207.)

FIG. 11A depicts left ventricular wall thickness at end-diastole in the contralateral remote area and infarct area from contrast CT images.

FIG. 11B depicts infarct to contralateral remote circumferential wall stress ratio.

FIG. 12A depicts representative contrast CT images from control, MI-saline, MI-HA, and MI-HA/rTIMP-3 groups with the corresponding endocardial (red) and epicardial (green) contours.

FIG. 12B depicts decrease in the average peak circumferential strain in the MI area for MI-Saline group compared to contralateral normal region.

FIG. 13A depicts representative dual isotope in vivo ²⁰¹T1/^{99m}Tc-RP805 SPECT/CT images from control, MI-Saline, MI-HA, and MI-HA/rTIMP3 pigs.

FIG. 13B depicts the ratio of ^{99m}Tc-RP805 in infarcted region relative to contralateral normal tissue in control and test subjects.

FIG. 14 depicts representative 3D SPECT/CT qualitative images of ex vivo hearts.

FIG. 15A depicts gamma well counting of left ventricular myocardial activity based on ²⁰¹T1 uptake in MI region compared to remote area in another MI-pigs.

FIG. 15B depicts myocardial ^{99m}Tc-RP805 uptake in MI region compared to remote area of MI-Saline pigs (p<0.001).

FIG. 15C depicts myocardial ^{99m}Tc-RP805 activity versus peak circumferential strain in MI region.

FIG. 16A depicts gamma well counting of left atrial activity based on ^{99m}Tc-RP805 uptake in the MI-HA/rTIMP-3 group compared to the MI-Saline group.

FIG. 16B depicts left atrial diastolic volumes indexed to body weight in the MI-HA/rTIMP-3 group compared to the MI-Saline group.

FIG. 16C depicts left ventricle end diastolic pressure versus left atrial volume.

Repeat use of reference characters in the present specification and drawings is intended to represent the same or analogous features or elements of the present invention.

### DETAILED DESCRIPTION

Reference will now be made in detail to exemplary embodiments of the disclosure. It is to be understood by one of ordinary skill in the art that the present disclosure is a description of exemplary embodiments only and is not intended as limiting the broader aspects of the present disclosure.

The present disclosure is directed in part to a method of imaging a subject's lung. Methods disclosed herein include administering to a subject's lung an imaging agent labeled with a radioisotope, acquiring an image of the subject's lung using hybrid digital single-photon emission computed tomography (SPECT)/computed tomographic (CT) imaging, and measuring an amount of the imaging agent bound to matrix metalloproteinase in the subject's lung. Surprisingly, non-invasive methods disclosed herein allow for absolute quantification of an imaging agent's (e.g., ^{99m}Tc-RP805) uptake in a specific tissue (e.g., lung, heart, etc.).

In accordance with another embodiment, the present disclosure is directed to a method of evaluating a pulmonary infection in a subject. The method includes administering an imaging agent comprising a ^{99m}Tc-labeled matrix metalloproteinase targeted radiotracer to a subject's lung; acquiring an image of the subject's lung using SPECT/CT imaging; and measuring an amount of the imaging agent bound to matrix metalloproteinase in the subject's lung. The presence of the imaging agent bound to a matrix metalloproteinase in an amount higher than an amount in a reference subject indicates the subject is afflicted with a pulmonary infection.

In accordance with another embodiment, the present disclosure is also directed to a method of enhanced, quantitative high resolution imaging of a subject's heart tissue. The method includes administering to the subject's heart tissue an imaging agent comprising a ^{99m}Tc-labeled matrix metalloproteinase targeted radiotracer; acquiring an image of the subject's heart tissue using SPECT/CT imaging; and quantifying an amount of the imaging agent bound to matrix metalloproteinase in the subject's heart tissue (or localized to the heart tissue of the subject).

Surprisingly, methods disclosed herein may be utilized for novel clinical application of in vivo ^{99m}Tc-RP805 SPECT/CT imaging of tissues (e.g., lung tissue or heart tissue) for diagnosis and risk stratification of patients with viral or bacterial infections or a vascular disease. For instance, methods disclosed herein may be utilized for imaging lung tissue in a subject suspected of or at risk for acute respiratory distress syndrome (ARDS). This approach would have specific relevance for management of patients with COVID-19 infection.

Methods disclosed herein may allow for effective triage of patients presenting to the intensive care unit (ICU) with pulmonary infection. Since MMP activation is critically involved with disease progression, serial imaging could guide therapies directed at inhibition of MMPs and improve clinical outcomes. Further, imaging methods according to the present disclosure may facilitate application of different treatment options to reduce ICU requirements and decrease costs. More specific to the current COVID-19 health crisis, MMP imaging may provide an objective means to stratify the risk of respiratory failure thus helping to optimize how critical resources are allocated.

In one embodiment, the imaging agent may be labeled with a radioisotope. For instance, the radioisotope has imageable gamma-ray or positron emissions. The radioisotope may include, but is not limited to, 99mTc, 95Tc, 111In, 62Cu, 60Cu, 64Cu, 67Ga, 68Ga, or 86Y. For instance, the radioisotope may include 99mTechnetium (99mTc). The imaging agent disclosed herein may comprise a MMP targeted radiotracer. For instance, the imaging agent may be an MMP inhibitor. Binding of the MMP inhibitor in a tissue is indictive of MMP presence and/or activity. Thus, the activity of MMP can be measured or quantified based on the level of MMP inhibitor present (or localized) in an organ or tissue. MMP inhibitors disclosed herein exhibit selectivity for one or more MMPs. For instance, the MMP inhibitor may exhibit selectivity for MMPs including, but not limited to, MMP-1, MMP-2, MMP-3, MMP-9, MMP-12, MMP-13, or MMP-14. In one embodiment, the MMPs include MMP-2, MMP-9, or MMP-14. The MMP inhibitor binds to one or more MMPs for a period of time that is sufficient to detect their presence in the tissue being imaged. The imaging agent administered to a subject disclosed herein may include ^{99m}Tc-RP805, ^{99m}Tc-pyrophosphate (^{99m}Tc PYP), or ^{99m}Tc-Maraciclatide (^{99m}Tc-NC100692, GE Healthcare) (see Dearling et al. 2013, Nucl Med Biol.40:788-94). In one embodiment, the imaging agent is ^{99m}Tc-RP805, see U.S. Pat. No. 6,656,448 and as described in greater detail herein.

According to the present disclosure, an SPECT/CT image of a ^{99m}Tc-labeled matrix metalloproteinase targeted radiotracer can be acquired simultaneously with a second imaging agent. This simultaneous dual isotope imaging can be done by utilizing radioisotopes of the MMP and perfusion imaging agents which have spectrally separable gamma emission energies. For instance, the second imaging agent may be ²⁰¹Thallium (²⁰¹T1) or any other imaging agent that is or that comprises I-123, In-111, xenon-133, or gallium-67. In one embodiment, for myocardial perfusion images, dual isotope SPECT/CT imaging may be performed with ^{99m}Tc-RP805 and ²⁰¹T1. This is possible because the ^{99m}Tc-RP805 gamma energy of ~140 kV and the ²⁰¹T1 gamma energy of −70 kV are easily separable from each other. This simultaneous imaging of cardiac perfusion and extracellular matrix degradation (as evidenced by MMP inhibitor localization) is useful for improved anatomic assessment of the MMP imaging agent localization and distribution in the heart tissue of a subject based on the comparison to the perfusion distribution seen in the ²⁰¹T1 image. In addition, the simultaneous imaging of perfusion and extracellular matrix degradation allows a more complete assessment of the underlying cardiac disease, both in terms of blood flow alterations and biochemical changes, in a single imaging session on a patient.

The simultaneous dual-isotope imaging of cardiac perfusion and extracellular matrix degradation allows the localization of sites of vulnerable plaque and cardiac perfusion to be visualized during one imaging session. In addition, the simultaneous imaging of tissue changes associated with congestive heart failure (from the MMP imaging agent) and coronary artery disease (from the perfusion imaging agent) is useful in characterizing the underlying causes of congestive heart failure.

According to the present disclosure, SPECT/CT images may be acquired in combination with an echocardiogram (ECHO) or contrast computed tomography (CCT).

ECHO provides real-time single, two-dimensional, and three-dimensional views of the heart. CCT can also provide the same functional information. From these images, regional ventricular wall motion and thickness is determined. Myocardial infarction causes abnormalities in regional wall motion, which can be assessed by echocardiography or CCT. Additionally, echocardiographic contrast agents can be used to assess regional blood flow.

Subjects to which an imaging agent disclosed herein can be administered include mammals, such as primates, for example humans. For veterinary applications, a wide variety of subjects are suitable, e.g., livestock such as cattle, sheep, goats, cows, swine, and the like; poultry such as chickens, ducks, geese, turkeys, and the like; and domesticated animals, such as pets including dogs and cats. For diagnostic or research applications, a wide variety of mammals are suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject. Tissue in which the imaging agent is administered to may include, but is not limited to, lung, heart, blood, liver, spleen, kidney, bone, muscle, etc. In one embodiment, the tissue in which the imaging agent is administered is lung tissue.

An imageable amount of the imaging agent may be administered to a subject via routes of administration disclosed herein for experimental, diagnostic, prophylactic, and/or therapeutic purposes. For instance, the imaging agent may be administered to the subject via parenteral administration. “Parenteral administration” refers to administration of the imaging agent via a route in which a tissue of a subject is physically breached, such as subcutaneous, intravenous, intraperitoneal, intramuscular, intrasternal injection, and kidney dialytic infusion techniques. Solutions or suspensions used for parenteral administration, such as intradermal or subcutaneous administration, can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The subject disclosed herein may be at risk of, or suspected of being afflicted with, or diagnosed with a disease or disorder, such as a viral or bacterial infection. In one embodiment, the subject is suspected of being afflicted with a pulmonary disease affecting the lungs and respiratory system. “Pulmonary diseases” as used herein include, but are not limited to, chronic obstructive pulmonary disease, pulmonary fibrosis, asthma, pulmonary hypertension, lung inflammation, and lung infection. For instance, the subject may be suspected of being afflicted with acute respiratory distress syndrome (ARDS), coronavirus disease 2019 (COVID-19), chronic obstructive pulmonary disease (COPD), lymphangioleiomyomatosis (LAM), idiopathic pulmonary fibrosis (IPF), or acute lung injury (ALI). In another embodiment, a subject may be suspected of being afflicted with a vascular disease including ischemic stroke, hemorrhagic stroke, transient ischemic attack (TIA), vascular inflammation due to meningitis, atherosclerosis, arteritis, physical obstruction of arterial blood supply to the brain, lacunar stroke, hypoperfusion embodying diffuse injury, myocardial infarction and arrhythmia, restenosis associated with percutaneous transluminal coronary angioplasty, peripheral vascular disease and cerebral vascular disease, venous occlusive disorders such as deep vein thrombosis, hypercoagulopathies, aneurysms or chronic progressive vascular disease.

MMPs play a role in lung pathologies through their ability to process extracellular matrix proteins and non-matrix mediators of lung damage (e.g., cytokines, chemokines, and surface receptors). Growth factors, cytokines, and chemokines are believed to influence the expression and secretion of MMPs in both lung parenchymal and non-parenchymal cells in pulmonary conditions. For instance, MMP-1, MMP-2, MMP-3, MMP-7, MMP-8, MMP-9, MMP-11, MMP-12, and MMP-14 contribute to COPD-associated pulmonary damage and ARDS. It is understood that each MMP plays an individual role during specific periods in the progression of any given pulmonary pathology.

In one embodiment, the subject is suspected of being afflicted with ARDS. ARDS is characterized by persistent pulmonary neutrophilic inflammation, edema, and pulmonary hemorrhage. ARDS patients typically require assisted ventilation with positive pressure.

MMPs have been implicated in the pathogenesis of ARDS since lung levels of several MMPs are increased in patients with ALI and ARDS, and lung levels of some MMPs correlate positively with adverse clinical outcomes in ARDS patients. For instance, bronchoalveolar lavage (BAL) fluid (BALF) levels of collagenases (MMP-1, −8, and −13), gelatinases (MMP-2 and −9), and stromelysin-1 (MMP-3) are elevated in patients with ALI and ARDS. Thus, increased levels of MMPs serve as a biomarker for ARDS. Methods disclosed herein may be utilized to image and quantify the amount of ^{99m}Tc-RP805 bound to MMP in a subject's lung tissue to diagnose ALI/ARDS.

In one embodiment, the subject is suspected of being afflicted with COVID-19. COVID-19, formerly known as the 2019 novel coronavirus, is caused by severe acute respiratory syndrome coronavirus 2 (SARS-CoV-2). Elevated MMP levels in a subject are believed to be an immune-based biomarker of COVID-19. MMP-9 has been implicated as a participant in ALI/ARDS, specifically in patients suffering from asthma, pulmonary fibrosis, and COPD. Thus, elevated MMP-9 levels in a patient afflicted with COVID-19 may result in respiratory failure. Production of MMP-9 likely involves the coronavirus-induced MMP-9 release from neutrophils, which promote the inflammation and degradation of the alveolar capillary barrier. Methods disclosed herein may be utilized to image and quantify the amount of ^{99m}Tc-RP805 bound to MMPs, such as MMP-9, in a subject's lung tissue to diagnose COVID-19 or to monitor or assess the progression of the infection and/or its sequalae or other lung condition.

The present disclosure contemplates, in part, measuring the presence and in some instances amount (or level) of an administered imaging agent (e.g., an MMP inhibitor) and comparing this to a control in order to determine an increased risk of developing a pulmonary infection. Methods disclosed herein intend to determine an “above-normal” or “above-average” or “increased” risk of a pulmonary infection or other indication. An above-normal or above-average risk or increased risk is a risk that is greater than the risk of a normal subject or a population of normal subjects or a randomly selected population for developing ARDS or other indications. In some instances, an above-normal or above-average risk or increased risk is indicated by any level of imaging agent bound to MMP that is greater than the level of imaging agent bound to MMP of a reference subject or population. In some instances, the increased risk is further quantified by measuring the imaging agent level bound to MMP, wherein relatively lower imaging agent levels indicate a lower “increased” risk and relatively higher imaging agent levels indicate a higher “increased” risk, provided that even the lower imaging agent levels are still above normal or control levels.

The control level may be an imaging agent level bound to MMP determined using the same imaging agent in a reference subject (i.e., a subject that is known not to have a pulmonary infection), or it may be the average imaging agent level in a population of reference subjects, or it may be the average imaging agent level in a random sampling of the population at large. The control level may be one that is determined prior to the analysis of the subject rather than one that is determined in real time. The control level may therefore be a level that is obtained and established on a periodic basis (e.g., every 6 months, every year, etc.).

Regardless of the control used, increased risk of developing a pulmonary infection may be indicated by the subject's tissue uptake of the imaging agent that is above a reference or control level, or it may be indicated by an imaging agent level in a subject's tissue that is at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700% or 800%, or 10-fold, 20-fold, 50-fold, 100-fold, 200-fold, 300-fold 400-fold, or 500-fold more than the control level. In one embodiment, the imaging agent uptake is from about 20% to about 800% more than said imaging agent uptake in a reference subject. It is noted that the degree of increase may be predictive of subsequent injury and outcomes caused by a pulmonary infection.

Regardless of the control used, increased risk of developing a vascular disease may be indicated by the subject's tissue uptake of the imaging agent that is above a reference or control level, or it may be indicated by an imaging agent level in a subject's tissue that is at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700% or 800%, or 10-fold, 20-fold, 50-fold, 100-fold, 200-fold, 300-fold 400-fold, or 500-fold more than the control level. In one embodiment, the imaging agent uptake is from about 20% to about 800% more than said imaging agent uptake in a reference subject. It is noted that the degree of increase may be predictive of subsequent injury and outcomes caused by a vascular disease.

According to the present disclosure, the imaging agent (e.g., ^{99m}Tc-RP805) may be administered to a subject at a total dose of from about 2 mCi to about 50 mCi, such as from about 5 mCi to about 45 mCi, such as from about 10 mCi to about 35 mCi, such as from about 15 mCi to about 30 mCi, or any range therebetween.

According to the present disclosure, the perfusion imaging agent (e.g., ²⁰¹T1) may be administered to a subject at a total dose of from about 0.5 mCi to about 5 mCi, such as from about 1.0 mCi to about 4.5 mCi, such as from about 1.5 mCi to about 4 mCi, such as from about, 2.0 mCi to about 3.5 mCi, or any range therebetween. The perfusion agent may be administered prior to, concurrent, or subsequent to the administration of the ^{99m}Tc-labeled matrix metalloproteinase targeted radiotracer. In one embodiment, the perfusion agent is administered at from about 200 minutes to about 300 minutes after administration of the radiotracer, such as after about 240 minutes, such as after about 270 minutes, such as after about 300 minutes. In another embodiment, the perfusion agent is administered concurrently with the imaging agent. In another embodiment, the perfusion agent is administered at from about 200 minutes to about 300 minutes prior to administration of the radiotracer, such as prior to about 240 minutes, such as prior to about 270 minutes, such as prior to about 300 minutes According to the present disclosure, images of a subject (including images of a region or tissue or organ of a subject) may be acquired post-administration of the imaging agent after a period of time to allow for biodistribution of the imaging agent. Images may be acquired using single-photon emission computed tomography (SPECT) imaging, cine computed tomographic (CT) imaging, positron emission tomography (PET), digital hybrid SPECT/CT imaging, magnetic resonance imaging (MRI), or ultrasound. For instance, images are acquired using digital hybrid SPECT/CT imaging, such as nano-SPECT/CT imaging in one embodiment. In another embodiment, images are acquired on a hybrid CZT SPECT/64-slice CT scanner (Discovery 570 NM/CT, GE Healthcare). The imaging detector, for instance, may be a semiconductor detector such as a cadmium-telluride (CdTe), cadmium-zinc-telluride (CZT), or a mercuric-iodide (Hgl) detector. Images may be acquired upon injection or at about 60 minutes to about 300 minutes after administration of the imaging agent, such as after about 90 minutes, such as after about 120 minutes, such as after about 150 minutes, such as after about 180 minutes, such as after about 210 minutes, such as after about 240 minutes, such as after about 270 minutes, such as after about 300 minutes. Images may be acquired prior to about 500 minutes, such as prior to about 400 minutes, such as prior to about 300 minutes, such as about 200 minutes, such as about 100 minutes, such as about 60 minutes.

According to the present disclosure, images acquired may comprise images of the subject's lung tissue including activity in parenchymal (i.e., alveolar), airway wall, vascular wall (e.g., endothelial), and immune cells, as well as blood and water (i.e., extracellular fluid). The image may comprise a subject's left lung. In another embodiment, the image may comprise a subject's right lung. In some embodiments, the image may comprise a subject's left and right lung. In one embodiment, a subject's image may comprise heart tissue including atrial myocardium (e.g., left atrial myocardium).

According to the present disclosure, imaging data acquired may be a two-dimensional or three-dimensional volume of data representing the localization of an imaging agent in a subject's tissue. “Imaging data” as used herein refers to a plurality of scalar values representing different locations in a Cartesian or polar coordinate format different than an actual display image. For instance, the imaging data may be a plurality of red, green, and blue values output to a display for generating the image in the display format on a scanner. The imaging data includes a dataset used for imaging, such as scan data.

According to the present disclosure, the imaging data may represent a two-dimensional or three-dimensional region of the subject. For example, the imaging data represents an area or slice of the patient as pixel values. In one embodiment, the imaging data represents a volume or three-dimensional distribution of voxels. The three-dimensional representation may be formatted as a stack or plurality of two-dimensional planes or slices. Values are provided for each of multiple locations distributed in two or three dimensions. The imaging data are acquired as one or more frames of data. The frame of data represents the scan region at a given time or period. The dataset may represent the area or volume over time, such as providing a 4D representation of the subject.

Methods disclosed herein contemplate quantification of ^{99m}Tc-RP805 using hybrid digital SPECT/CT imaging without and with CT contrast agents. If desired, a myocardial perfusion imaging agent may be utilized to determine myocardial perfusion (e.g., blood flow through the heart). For instance, a measure of myocardial perfusion is used together with quantification of ^{99m}Tc-RP805 uptake. Qualitative assessment of images can be completed using commercial system software (e.g., AW Workstation, GE Healthcare). For instance, Patlak graphical analysis can be applied to the imaging to quantify the imaging agent uptake. The slope of the Patlak model is a useful quantitative index that characterizes the net influx rate of the tracer into a region of interest (ROI). Alternatively, a static image quantification approach can be applied (e.g., standard uptake value). For example, the use of a standard uptake value (SUV), which is the concentration measured within a region or voxel normalized to the patient weight and the injected activity. SUV is affected by air within ROIs; therefore normalizing the SUV for the air fraction will likely improve the accuracy of estimation of activity with lung cells.

MMP inhibitors disclosed herein preferentially accumulate in the regions of the myocardium involved in myocardial and vascular injury and fibrosis and repair associated with atrial and ventricular remodeling and have been associated with risk for atrial or ventricular arrhythmias. The myocardial perfusion imaging agent may include, but is not limited to, Thallium-201 or Tc-Sestamibi which each accumulate in myocardium with normal blood perfusion, and demonstrate no or reduced uptake in ischemic myocardium under stress or in the presence of myocardial infarction. In another embodiment, the myocardial perfusion imaging agent is flurpiridaz F18, described in U.S. Pat. No. 7,344,702, having the following structure

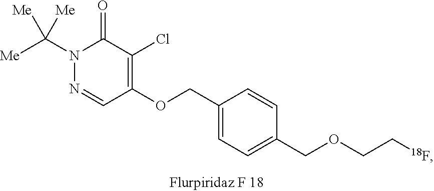

and which is known to cumulate in normal, viable and hibernating myocardium, in the presence of glucose and insulin, and have no or reduced uptake in chronically infarcted tissues.

The MMP-specific imaging agents of this disclosure include RP-805 and analogs thereof. Accordingly, it is to be understood that the disclosure contemplates use of RP-805 analogs in place of RP-805 in the various methods and applications provided herein.

RP805 has the following structure:

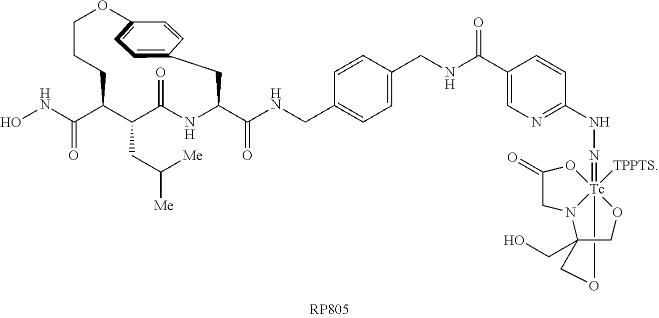

wherein TPPTS is 3,3′,3″-Phosphanetriyltris(benzenesulfonic acid)trisodium salt.

An example of another MMP-specific imaging agent contemplated by this disclosure is RP-782 which has the following structure:

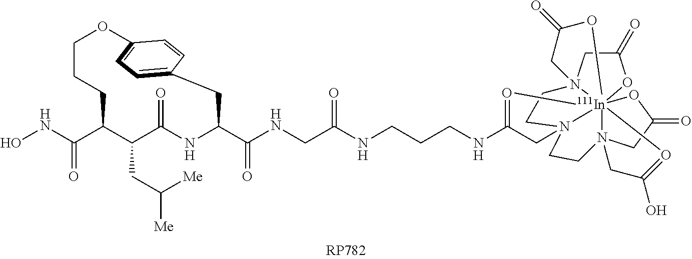

RP805 analogs may comprise
i) 1-10 targeting moieties;
ii) a chelator; and
iii) 0-1 linking groups between the targeting moiety and chelator;
   wherein the chelator is capable of conjugating to a diagnostic metal, and wherein the targeting moiety is a matrix metalloproteinase inhibitor of the formulae (Ia) or (Ib):

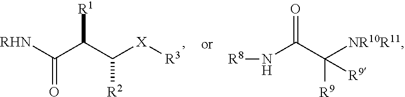

wherein
R is independently OH or —CH₂SH;
R¹ is independently selected at each occurrence from the group: H, OH, C₁₋₃ alkyl, C₂₋₃ alkenyl, C₂₋₃ alkynyl, and heterocycle-S—CH₂—;
R² is independently C₁₋₂₀ alkyl;
X is independently C═O or SO₂, provided when X is C═O, R³ is

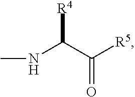

and
when X is SO₂, R³ is independently selected from the group: aryl substituted with 0-2 R⁶, and heterocycle substituted with 0-2 R⁶;
R⁴ is independently selected at each occurrence from the group: C₁₋₆ alkyl, phenyl, and benzyl;
R⁵ is independently selected at each occurrence from the group: NH(C₁₋₆ alkyl), NH-phenyl, and NH-heterocycle; wherein said alkyl, phenyl and heterocycle groups are optionally substituted with a bond to the linking group or a bond to the chelator;
R⁶ is independently aryloxy substituted with 0-3 R⁷;
R⁷ is independently halogen or methoxy;
or alternatively,
R¹ and R⁴ may be taken together to form a bridging group of the formula —(CH₂)₃-O-phenyl-CH₂—, optionally substituted with a bond to the linking group or a bond to the chelator; or alternatively,
R¹ and R² may be taken together to form a bridging group of the formula —(CH₂)₃—NH—, optionally substituted with a bond to the linking group or a bond to the chelator; or
R¹ and R² taken together with the nitrogen and carbon atom through which they are attached form a C₅₋₇ atom saturated ring system substituted with one or more substituents selected from the group consisting of: a bond to the linking group, a bond to chelator, and —C(═O)—NR²⁹R³⁰;
R⁸ is independently selected at each occurrence from OH or phenyl, optionally substituted with a bond to the linking group or a bond to the chelator, provided that when R⁸ is phenyl, R¹⁰ is —C(═O)—CR¹²—NH—CH(CH₃)—COOH;
R⁹ and R^{9′} are independently H, C₁₋₆ alkyl optionally substituted with a bond to the linking group or a bond to the chelator, or are taken together with the carbon atom to which R⁹ and R^{9′} are attached to form a 5-7 atom saturated, partially unsaturated or aromatic ring system containing 0-3 heteroatoms selected from O, N, SO₂ and S, said ring system substituted with R⁶ and optionally substituted with a bond to the linking group or a bond to the chelator;
R¹⁰ and R¹¹ are independently H, or C₁₋₆ alkyl optionally substituted with a bond to the linking group or a bond to the chelator, or are taken together with the nitrogen atom to which they are attached to form a 5-7 atom saturated, partially unsaturated or aromatic ring system containing 0-3 heteroatoms selected from O, N, SO₂ and S, said ring system optionally substituted with 0-3 R²⁷, a bond to the linking group or a bond to the chelator;
   or alternatively,
R⁹ and R¹⁰ are taken together with the carbon atom to which they are attached to form a 5-7 atom saturated, partially unsaturated or aromatic ring system containing 0-3 heteroatoms selected from O, N, SO₂ and S, said ring system optionally substituted with a bond to the linking group or a bond to the chelator; and
R¹² is independently C₁₋₂₀ alkyl;
R²⁷ is ═O, C₁₋₄ alkyl, or phenyl substituted with R²⁸;
R²⁸ is a phenoxy group substituted with 0-2 OCH₃ groups;
R²⁹ and R³⁰ taken together with the nitrogen atom through which they are attached form a C₅₋₇ atom saturated ring system substituted with R³¹; and
R31 is a benzyloxy group substituted with C₁₋₄ alkyl.

In some embodiments, the diagnostic metal is selected from the group consisting of a paramagnetic metal, a ferromagnetic metal, a gamma-emitting radioisotope, or an x-ray absorber. In some embodiments, the diagnostic metal is radioisotope selected from the group consisting of ^{99m}Tc, ⁹⁵Tc, ¹¹¹In, ⁶²Cu, ⁶⁴Cu, ⁶⁷Ga, and ⁶⁸Ga, optionally wherein the radioisotope is ^{99m}Tc, or optionally wherein the radioisotope is ¹¹¹In

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

The term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Methods

rTIMP-3 Formulation and Hydrogel (e.g., Hyaluronic Acid Hydrogel) Synthesis

The rTIMP-3 and hydrogel formulation (hydrolytically degradable HA hydrogel) was synthesized and validated (Polymeric Biomaterials Laboratory, University of Pennsylvania) for sustained release of rTIMP-3 at a targeted concentration over the 14-day period following intramyocardial injection.

### Myocardial Infarction and Hydrogel Delivery

Mature male Yorkshire pigs (n=19), 20-25 kg, were anaesthetized (ketamine/acepromazine/atropine: 22/1.1/0.04 mg/kg IM, respectively), intubated and then maintained on 1.5-2% isoflurane delivered in an oxygen/nitrous mixture (3:1 L/min, respectively). Buprenorphine (0.05 mg/kg IM) was administered as pre-surgery analgesia. Pigs underwent a small left lateral thoracotomy to expose the LV for surgical occlusion of the first two obtuse marginal arteries of the circumflex arteries (OM1 and OM2) to create a lateral wall MI. A 2×2 cm² grid was applied below the circumflex artery providing a layout for the nine injection sites in 3×3 matrix [4] for post-MI intramyocardial delivery of saline (MI-saline, n=8), HA alone (MI-HA, n=5), or HA that released rTIMP-3 (MI-HA/rTIMP-3, n=6). A transdermal fentanyl patch was placed for post-operative analgesia. Approximately one-week post-surgery, animals were transported in climate-controlled vehicles from the University of South Carolina to the Yale Translational Research Imaging Center where, after an additional 3-7 days of acclimatization, animals were utilized in terminal imaging procedures (10-14 days post-surgery). For imaging, general anesthesia was induced with ketamine 20 mg/kg and xylazine 10 mg/kg prior to intubation, and mechanical ventilation and anesthesia maintained with 1.5-2% isoflurane with a 35% oxygen/65% nitrous oxide mixture to ensure a surgical plane of anesthesia. A jugular vein was isolated, and a vascular sheath placed (6F, Cordis) for administration of fluids, and injection of radiotracers and contrast. A carotid artery was also isolated for placement of a sheath (7F, Cordis) for monitoring of arterial blood pressure.

A separate cohort of control pigs (n=7) that had no coronary intervention or intramyocardial injections, underwent imaging and were analyzed in identical fashion to establish referent normal values.

### In-vivo CT Imaging and Processing

In vivo non-contrast and contrast CT angiograms were acquired on a hybrid CZT SPECT/64-slice CT scanner (Discovery 570 NM/CT, GE Healthcare) with ECG gating under a surgical plane of anesthesia at end-expiration. Following a non-contrast CT scan, iodinated contrast (25-35 ml, Ominipaque (350 mg/ml iodine), was injected intravenously (3 ml/min with 30 ml saline flush) via a power injector (Medrad, Warrendale, Pa) and retrospective CT images acquired using 120 kV, 350 mA, with a reconstructed slice thickness of 0.625 mm. The ECG-gated contrast CT scans were reconstructed over the entire cardiac cycle in 10% phases using filtered back projection with a voxel size of 0.977 mm³.

The LV endocardial and epicardial surfaces from the contrast CT scans were segmented for calculation of LV ejection fraction along with left atrial end-diastolic (corresponding to LV systole) volumes using commercial software (AW Workstation, GE Healthcare). The contrast CT images were also reconstructed into 3 mm thickness short axis slice and semi-automatically contoured using a commercial software (Segment CT, Medviso, Sweden). The contours were manually adjusted as required across all slices from base to apex. The software uses deformable image registration to track the displacement of the myocardium over the cardiac cycle. Strain values are reported as peak circumferential using the 17-segment AHA model with the apex removed. End-diastolic wall thickness was calculated by the software from endo- and epicardial contours. The mid-inferolateral region was analyzed as representative of the central infarct region and compared to the normal contralateral remote mid-anterior and mid-anteroseptal walls. Mean circumferential wall stress for each region was calculated as follows:

$$σ = \frac{{Pr}^{2}}{2 t ( r + \frac{t}{2} )} , \qquad ( I )$$

where r is the inner LV radius (calculated at the level of the papillary muscles), and t is the end-diastolic wall thickness measured from the contrast CT images. P is the LV end-diastolic pressure acquired just prior to CT acquisitions using a micromanometer catheter (Millar Instruments) placed in the LV cavity via the carotid artery. Due to technical issues with LV pressure measurements, data were not acquired for 2 controls and 3 MI-Saline animals and are therefore not included in the wall stress calculations.

### SPECT Imaging of Myocardial MMP Activation and Perfusion

Approximately 4-5 hrs following intravenous injection of ^{99m}Tc-RP805 (22-32 mCi, MicroVide, LLC), and 15 min following intravenous injection of ²⁰¹T1 (1.5-3 mCi), a dual isotope SPECT/CT image was acquired over 15 min on a hybrid solid state cadmium-zinc-telluride detector SPECT/64-slice CT scanner (Discovery 570 NM/CT, GE Healthcare) in list mode using 5% windows centered on the energy peaks (²⁰¹T1-70 kV; and ^{99m}Tc-140 kV). SPECT images were reconstructed using standard iterative algorithms for qualitative and quantitative assessment of in-vivo radiotracer uptake using CT-based attenuation correction. Quantitative assessment of ^{99m}Tc-RP805 uptake was completed by defining the LV region of interest (ROI) with the ²⁰¹T1 uptake image and applying that ROI to the ^{99m}Tc-RP805 image (Flowquant™). The maximum uptake in the mid-inferior and mid-anterior lateral walls was indexed to the contralateral uptake in the mid-inferior and mid-anterior septal walls. The maximum uptake was selected to reduce partial volume effects. In-vivo image data were not available for three MI animals for analysis. At completion of the in vivo imaging, pigs were euthanized, hearts excised and filled with a dental molding material (alginate type II, regular set, Henry Schein) to preserve heart shape. The casted hearts were then imaged (15 min) using the same dual isotope windows and hybrid SPECT/CT imaging system to achieve high-resolution motion and background free ex vivo imaging for optimal evaluation of the 3-dimensional distribution of MMP activation (^{99m}Tc-RP805) and myocardial perfusion (²⁰¹T1). Qualitative assessment of images was completed using commercial system software (AW Workstation, GE Healthcare).

### Tissue Gamma Well Counting

Following SPECT/CT imaging, the RV was removed, and the LV sliced into 4-mm thick short axis slices from base to apex. Each slice was photographed to determine MI size as a percent of total LV volume using Image-J. Each slice was then divided into 8 epicardial and endocardial radial subdivisions for gamma well counting. The left atrium was also sectioned for gamma well counting. Well counting data were not available for one of the control pigs. Regional myocardial radiotracer uptake was computed as the injected dose per gram of tissue (% ID/g) after correcting for background activity and radioactive decay. LV tissue segments with values greater than 70% of the overall maximum ²⁰¹T1 uptake in the LV were assessed to be normal regions, and segments with uptake with less than 40% of the maximum ²⁰¹T1 uptake were considered the central infarct region. These segmental designations were applied for analysis of myocardial ^{99m}Tc-RP805 uptake. To evaluate differences in left atrial uptake of ^{99m}Tc-RP805, the average atrial uptake was normalized to ²⁰¹T1 uptake.

### Statistical Analyses

All data are expressed as mean±standard deviation. The comparative analysis between the means of the controls, MI-saline, MI-HA and MI-HA/rTIMP-3 treated animals was assessed with a one-way ANOVA using post-hoc Tukey's analysis performed with GraphPad Prism. The comparison of means between contralateral normal and infarct area in the same group was assessed with a paired t-test performed in GraphPad Prism. A p-value less than 0.05 in both tests was considered significant.

### Lung Analysis

The following are exemplary imaging and modeling embodiments that may be used in lung analysis, including for example in the pig ARDS model.

Dynamic ^{99m}Tc-RP805 SPECT/CT scans may be acquired in list mode at 5 minutes to 15 minutes interval from 0 minutes to 120 minutes post radiotracer injection on a CZT SPECT 64-slice CT scanner (Discovery NMCT570c, GE). Regional and dynamic SPECT lung activity may be quantified using a semi-automatic total lung and lobar segmentation and intensity-based segmentation.

Tissue fraction effect from air may be corrected by radiometric estimation of air fraction from HU of air and soft tissue in attenuation correction CT scans. The fraction of blood flow in each voxel may be corrected by using the first 90 seconds of the initial dynamic acquisition. The SPECT images may be reconstructed for the estimation of signal from the lung parenchymal tissue.

### Example 1

Myocardial infarction (MI) often results in infarct thinning and expansion and left ventricular (LV) remodeling and can ultimately lead to heart failure (HF) and increased mortality. The induction of proteolytic enzymes in the myocardium, including the matrix metalloproteinases (MMPs), occurs early in patients following MI and is related to the degree of adverse LV remodeling. In evaluation of post-MI remodeling, the present inventors have previously established an imaging approach for assessment of regional MMP activation within the LV myocardium utilizing single-photon emission computed tomography (SPECT) imaging of a ^{99m}Tc-labeled MMP targeted radiotracer, ^{99m}Tc-RP805. The transition from traditional SPECT cameras with Na-Iodide detectors, photomultipliers, and rotating gantries to higher sensitivity and higher resolution solid state cadmium-zinc-telluride (CZT) detector systems enables creation of high resolution cardiac gated images from dynamic listmode datasets. There is a clear advantage in the clinical setting in the ability to assess LV function with early SPECT images and late “hotspot” images of MMP activation without the need for injection of other radiotracers or iodinated contrast CT. The purpose of this study was to evaluate early (0-5 min)^{99m}Tc-RP805 cardiac gated images for quantitative assessment of blood pool images for determination of left ventricular function in relation to late (4 hr) targeted “hotspot” images for quantification of LV MMP activity following an MI.

Dynamic ^{99m}Tc-RP805 images were retrospectively reconstructed for the initial 5 minutes of imaging in swine (n=5) 3 days following creation of reperfused infarction by 90-minute occlusion of the left anterior descending coronary artery. Pigs had dynamic imaging over 4 hours to assess late myocardial uptake of ^{99m}Tc-RP805. In each animal following the ECG-gated ^{99m}Tc-RP805 SPECT imaging, a high resolution (0.625 mm) ECG-gated contrast CT which was used as the gold standard for assessment of cardiac function was performed. A delayed enhancement CT was used for identification and co-localization of radiotracer activity in the MI region. The ECG-gated ^{99m}Tc-RP805 SPECT scans were analyzed with commercial software (4DM, Invia) and compared with ECG-gated contrast CT images (AW software, GE Healthcare) for determination of left ventricle ejection fraction (LVEF).

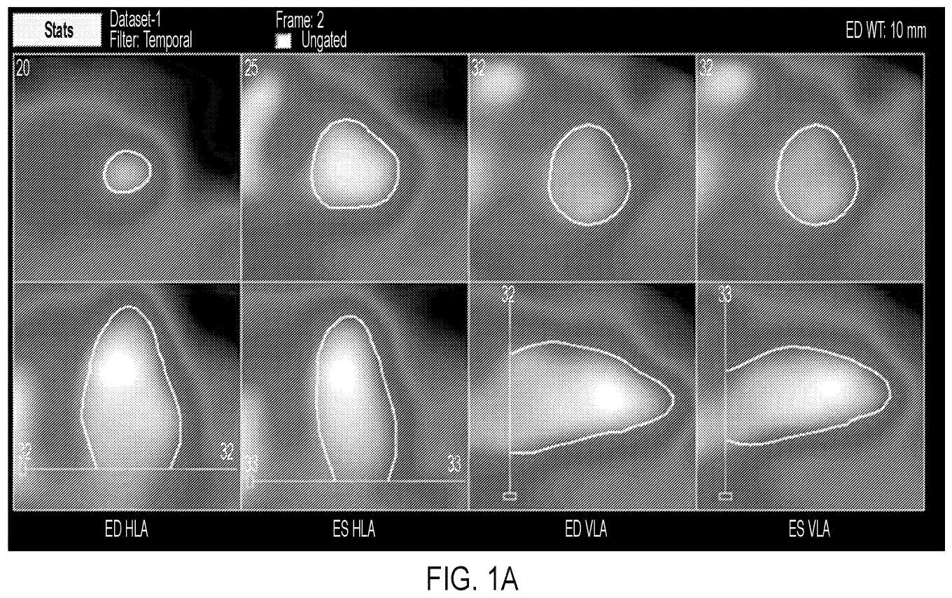

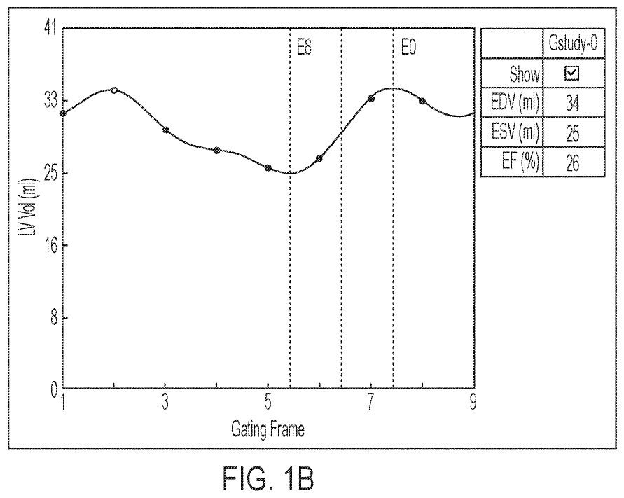

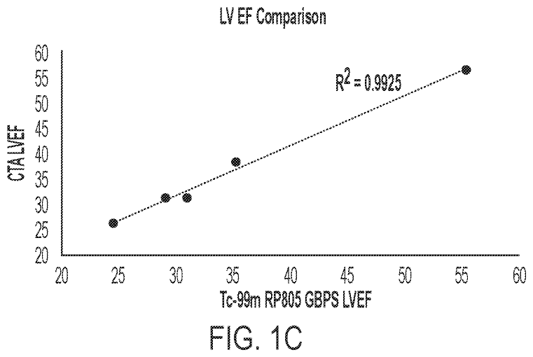

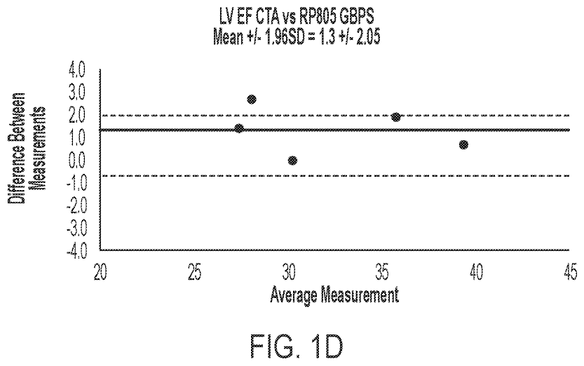

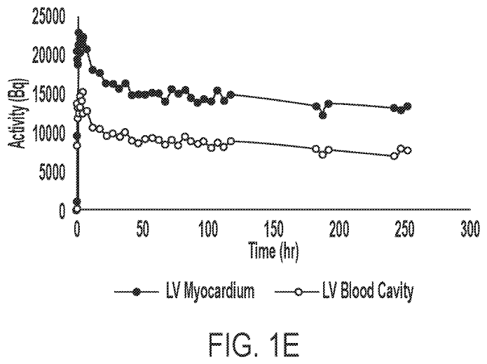

FIGS. 1A-E shows the excellent image quality obtained in representative initial ECG-gated ^{99m}Tc-RP805 images of blood pool activity and image analysis. There was a high correlation between the LVEF determined from ECG-gated contrast CT and the early ECG-gated ^{99m}Tc-RP805 SPECT blood pool with an R² of 0.9925 (FIG. 1C). A Bland-Altman analysis shows excellent agreement of the data and a low bias (FIG. 1D). A representative time activity curve displays the relative uptake in the global LV myocardium vs blood input function (FIG. 1E).

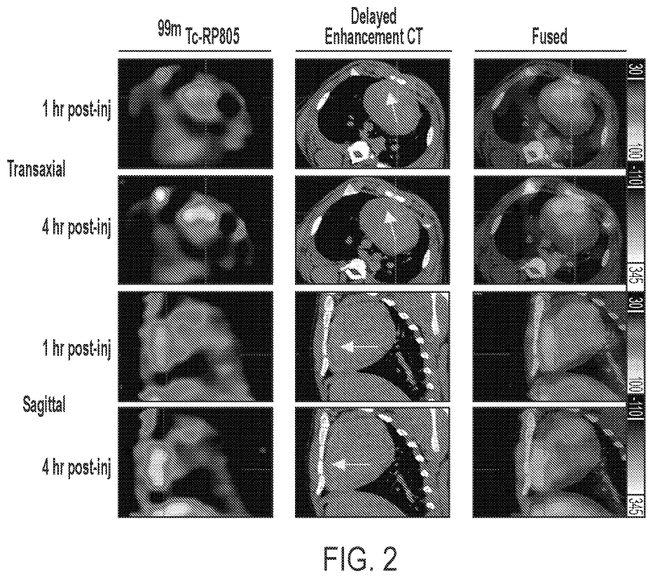

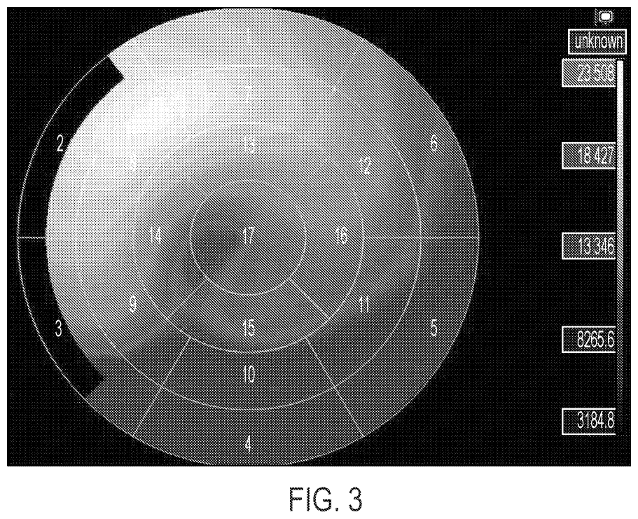

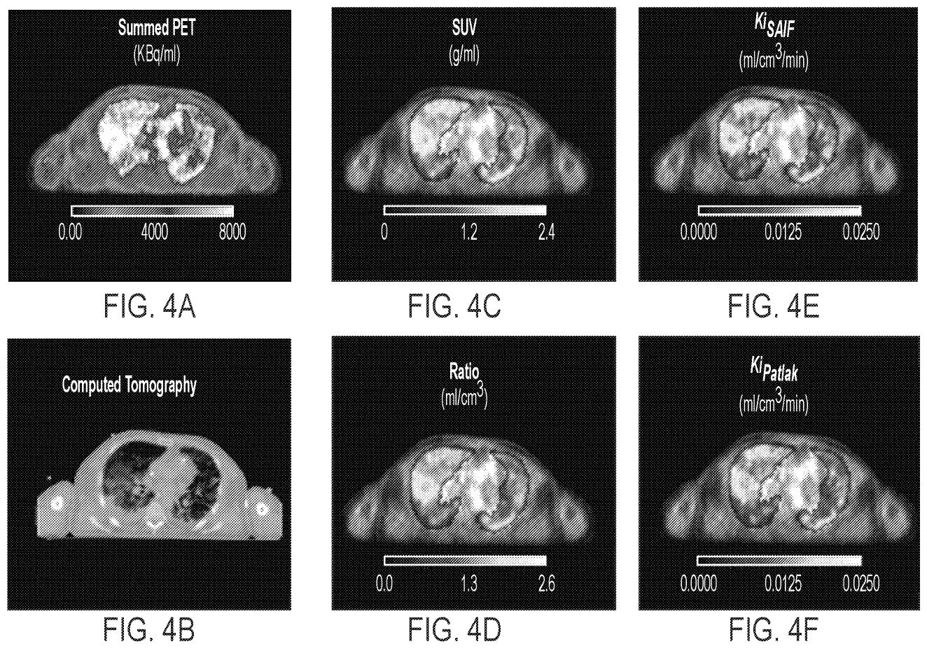

Focal uptake in the infarct area is clearly defined at 4 hr post-MI (FIG. 2). As seen in FIG. 2, there is clear definition of the MI region in the 4 hr image versus the 1 hr image. However, using the delayed enhancement CT (yellow arrow pointing to MI enhancement) to correctly delineate the focal uptake of ^{99m}Tc-RP805, quantification of myocardial activity in infarct area at 1 hr (37.04 counts/infarct area) was similar to 4 hr (37.58 counts/infarct area) when corrected for radiotracer decay.

While regional and global LV function can be derived from ECG-gated SPECT perfusion images, this is not possible with “hot spot” targeted MMP imaging using ^{99m}Tc-RP805. However, ECG-gated ^{99m}Tc-RP805 blood pool imaging provides a means to determine left ventricle function post-MI using initial blood pool images without additional radiotracers. Thus, important and clinically relevant data can be derived from initial bloodpool activity of a targeted “hotspot” imaging agent and correlated with focal radiotracer uptake in the myocardial infarct region.

### Example 2

Previous work has focused on quantification of relative myocardial ^{99m}Tc-RP805 uptake or myocardial uptake as a percentage of injected dose of radiotracer from static images. However, due to the unique technological developments of SPECT cameras, the present disclosure contemplates acquiring dynamic SPECT images for kinetic modeling and absolute radiotracer quantification, similar to dynamic quantitative PET imaging. Performing kinetic modeling of the dynamic ^{99m}Tc-RP805 images using a 2-tissue compartment model with 4 parameters seems to provide an excellent fit to the time activity curves (FIG. 1E). Our recent experimental data demonstrate there are no radioactive plasma metabolites formed out to 4 hour following ^{99m}Tc-RP805 injection. Using a 2-compartment model the present inventors have found imaging time can be shorten to 1 hour to achieve the same quantitation of tracer uptake that has been achieved with 4 hour delayed imaging that allows for radiotracer clearance from the blood pool. This technological development will allow more accurate quantification of the distribution volume of ^{99m}Tc-RP805 within the heart using only 1 hour of dynamic SPECT imaging.

### Example 3

### Quantification of RP805 Uptake in Lungs in ARDS

Acute respiratory distress syndrome (ARDS) is characterized by persistent pulmonary neutrophilic inflammation, edema, and pulmonary hemorrhage, which complicate quantitative analysis of SPECT imaging of the lungs with molecularly targeted radiotracers under this disease condition.

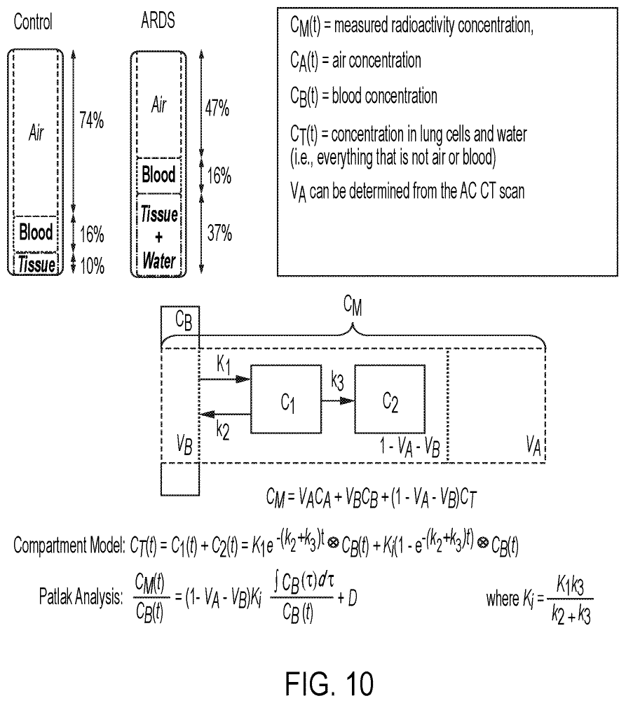

The signal within each SPECT voxel or predefined region of interest (ROI) in the lungs represents the contribution of activity in parenchymal (i.e., alveolar), airway wall, vascular wall (e.g., endothelial), and immune cells, as well as blood and water (i.e., extracellular fluid). Kinetic modeling of dynamic SPECT data can determine the fractional blood volume, V_{B}. SPECT and CT images have been used to estimate the regional air fraction (V_{A}). Using V_{A} and V_{B}, the radiotracer uptake in everything that is not air or blood (i.e., circulating cells and water) can be measured (see FIG. 10).

The lungs contain air, which is not the case for other organs. Therefore, an equation that accounts for air and blood fractions separately from the other lung components has been published and applied in idiopathic pulmonary fibrosis (IPF) and chronic obstructive pulmonary disease (COPD). See Chen et al. J. Nucl. Med. 2017 58(2): 201-207.

Patlak graphical analysis can be applied and is derived from the general compartment model for tracers that are irreversibly trapped in the target tissue like ^{99m}Tc-RP805. In conditions with increased edema, such as ARDS, the water fraction can be significant. See Patlak et al. J. Cereb. Blood Flow Metab. 1983 3:1-7; Patlak and Blasberg, J. Cereb. Blood Flow Metab. 1985, 5:584-590; Morris et al. Emission Tomography, 2004, Chapter 23, pages 499-540.

### Example 4

An alternative is to apply a static image quantification approach. For example, without wishing to be bound by theory, the use of a standard uptake value (SUV), which is the concentration measured within a region or voxel normalized to the patient weight and the injected activity. SUV is also affected by air within ROIs, therefore normalizing the SUV for the air fraction will likely improve the accuracy of estimation of activity with lung cells. Normalization for blood in ARDS such as with a tissue-to-blood ratio may further improve the accuracy of V_{B}.

Accurately matching tissue densities between SPECT and CT images is also essential for accurate SPECT image attenuation correction and V_{A} correction, which will require correction for respiratory motion correction. Changes in lung density from normal respiration between the SPECT and CT acquisitions can also lead to errors in attenuation and V_{A} correction.

In animal models of ARDS, the Patlak Ki correlated with ³H-deoxyglucose uptake in airway cells obtained by bronchoalveolar lavage, and Ki normalized for tissue fraction (determined independently by ¹³N-N₂ scans) correlated with lung neutrophil numbers by histology. Human studies in patients with ARDS have used the Patlak Ki without a correction for lung density or blood fraction but instead have simply compared the Ki in normal versus dense tissue separately across subjects. Dynamic [¹⁸F]FDG PET imaging was a valuable tool to study glucose metabolism in the inflamed lung. Fully kinetic modeling with spectral-based methods provides a unique insight into the pathophysiology of ALI, allowing a better understanding of the lung inflammation process and a more comprehensive description of the functional state of the tissue. [¹⁸F]FDG kinetics in human lung tissue can be described by an exponential model with two equilibrating components and one trapping component. The parameters derived can be correlated with the severity of the disease and can identify damaged areas by evaluating tissue-capillary integrity and tracer exchange rates.

A similar kinetic modeling for dynamic ^{99m}Tc-RP805 SPECT images of the lungs and chest in the setting of ARDS for risk stratification and for guidance of optimal ventilation schemes to minimize ventilator induced lung injury has been developed. This quantitative approach is possible with the recent availability of full field of view SPECT imaging systems with 360 degrees of solid state CZT detectors enabling high sensitivity dynamic SPECT imaging. This type of image quantification may require adjunctive non-contrast and contrast CT imaging for evaluation of tissue densities and lung aeration.

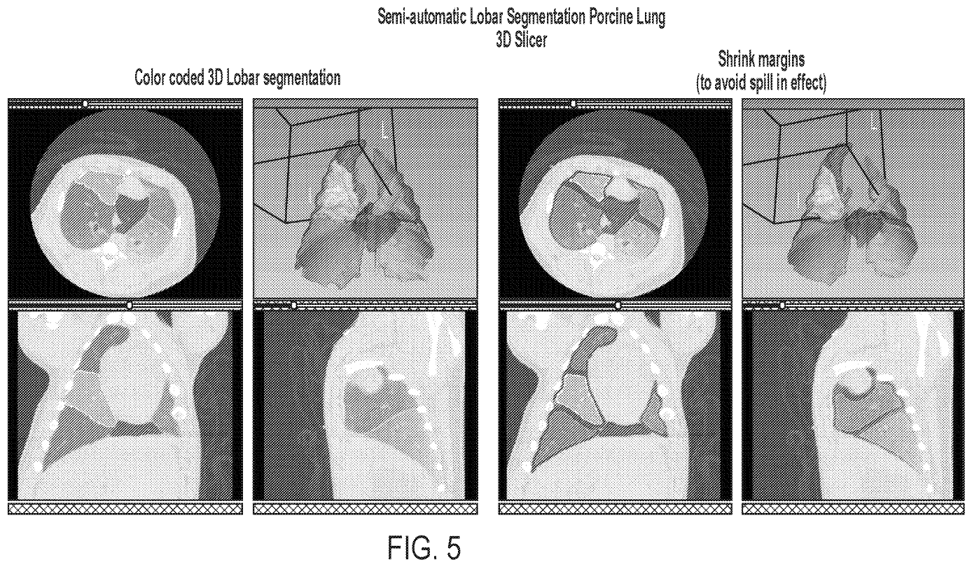

To apply quantitative analysis to lungs, the lobes may be segmented. An illustration of our 3D lobar segmentation is illustrated in FIG. 5, color coded segmentation shown (left). Volumes of interest (VOIs) were shrunken to avoid cross talk from adjacent structures outside of chest cavity (right).

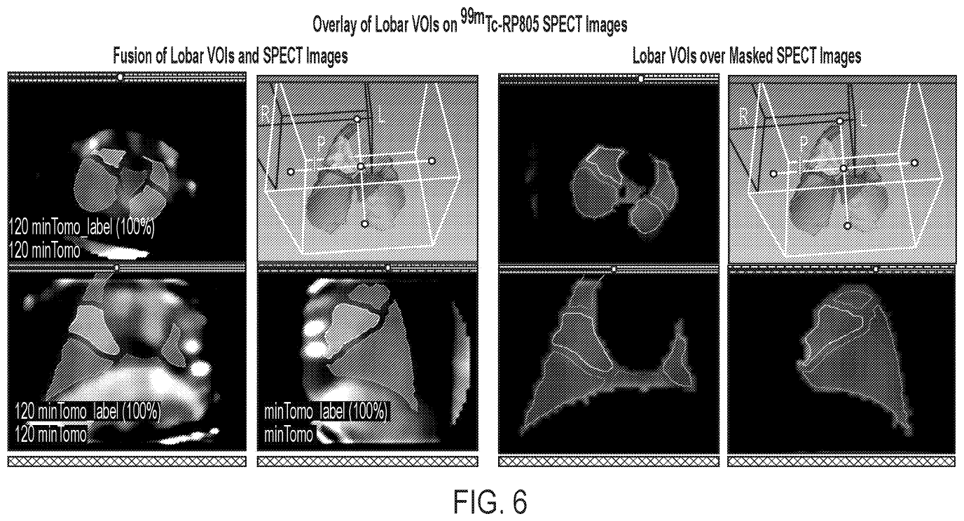

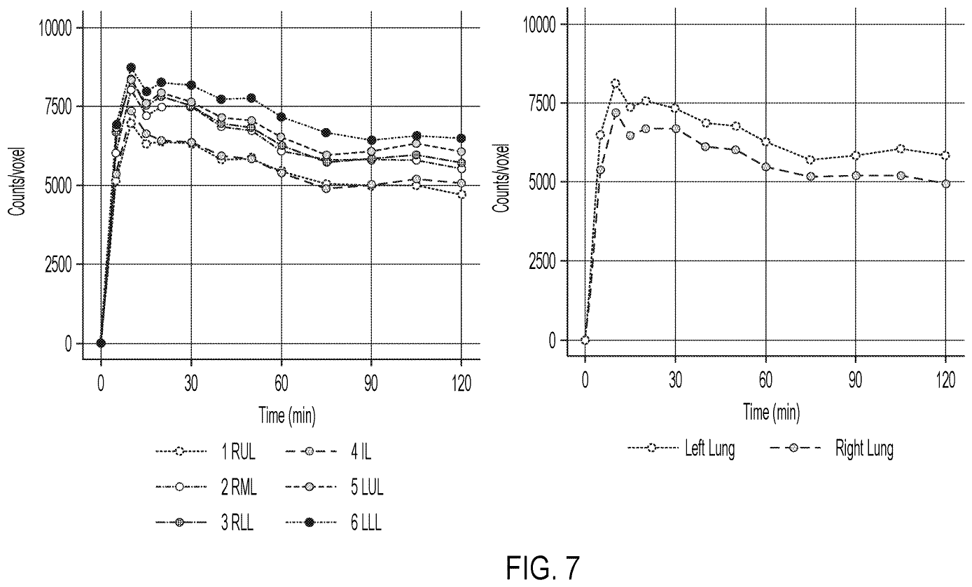

Shrunken segmented lobar VOIs are overlayed on dynamic ^{99m}Tc-RP805 SPECT images and images masked for lobar quantification of radiotracer activity as illustrated in FIG. 6. Regional quantification of ^{99m}Tc-RP805 uptake over 2 hours post injection in specific lobes and right and left lungs are illustrated in FIG. 7.

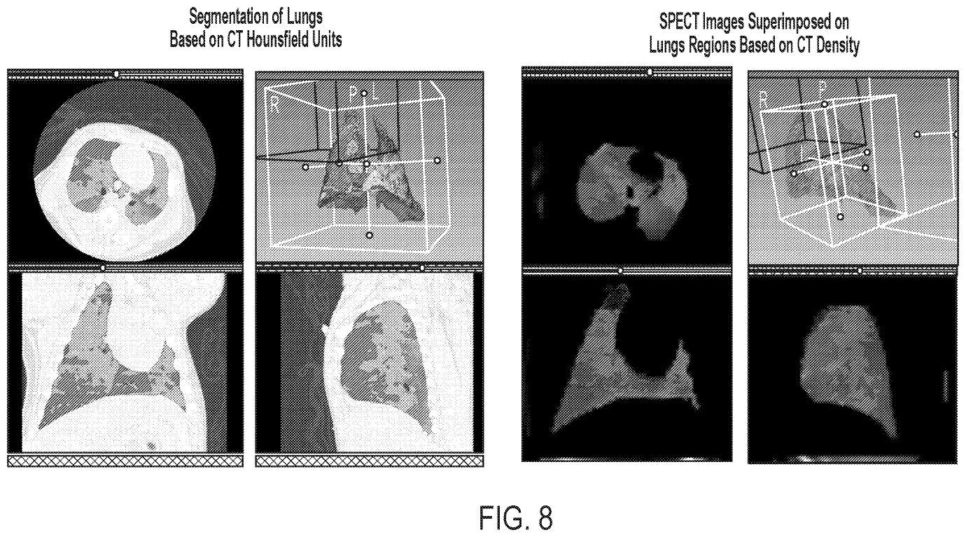

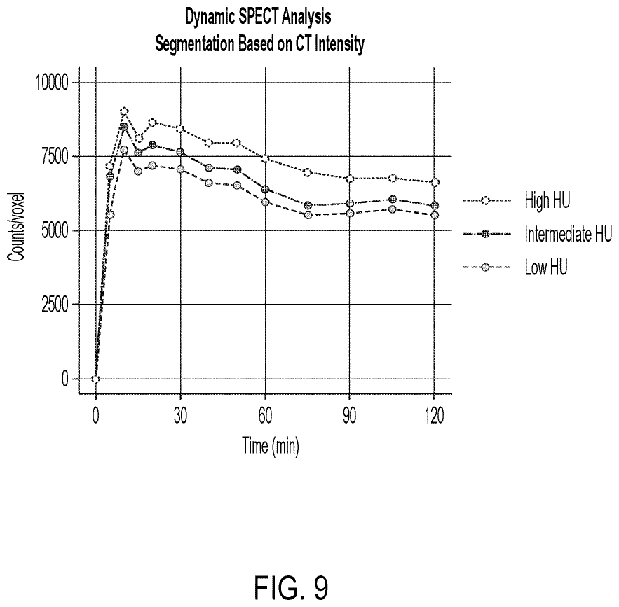

In addition to lobar analysis of dynamic ^{99m}Tc-RP805 SPECT images, analysis of lungs following lung injury was performed based on segmentation of the lungs according to ranges of Hounsfield density on non-contrast CT scans of chest (FIGS. 8 and 9).

### Example 5

### CT Assessment of Global LV Function and Regional Myocardial Thickness, Wall Stress, and Strain

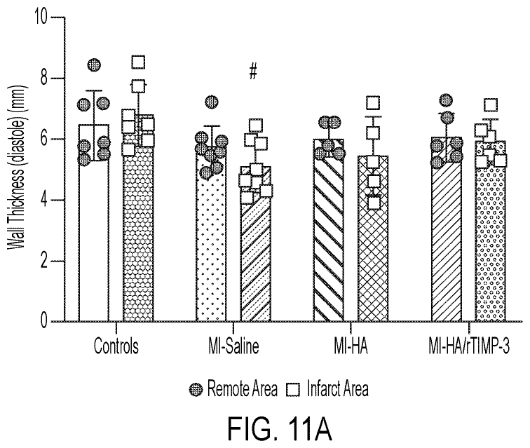

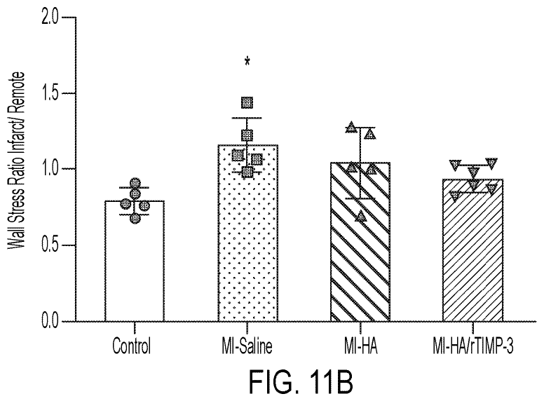

Global LV ejection fraction (% LVEF), LVEDV, regional wall thickness at end-diastole (ED), LV peak circumferential strains and circumferential wall stress were assessed in infarct and remote regions from contrast CT images. There was no significant difference in LVEF between groups at 10-14 days post-MI (MI-Saline: 37.41±11.10; MI-HA: 37.06±4.86; MI-HA/rTIMP-3: 34.73±12.20), although LVEF was uniformly lower than controls (46.98±14.90). There was a significant increase in LVEDV after correction for body weight as assessed with contrast CT imaging in the MI-Saline pigs compared to control (Control: 2.18±0.26 ml/kg; MI-Saline: 2.70±0.40 ml/kg, p<0.02), although a significant increase was not seen in either of the hydrogel groups (MI-HA: 2.18±0.23 ml/kg; MI-HA/rTIMP3: 2.47±0.23 ml/kg) relative to controls. There was a significant decrease in ED wall thickness in central MI region of the MI-saline pigs compared with referent controls (p=0.013) (FIG. 11A), although MI ED thickness was preserved in both the MI-HA and MI-HA/rTIMP-3 groups. Mean circumferential wall stress was increased in the MI area relative to the contralateral normal zone only in the MI-Saline group compared to controls (p=0.008) (FIG. 11B).

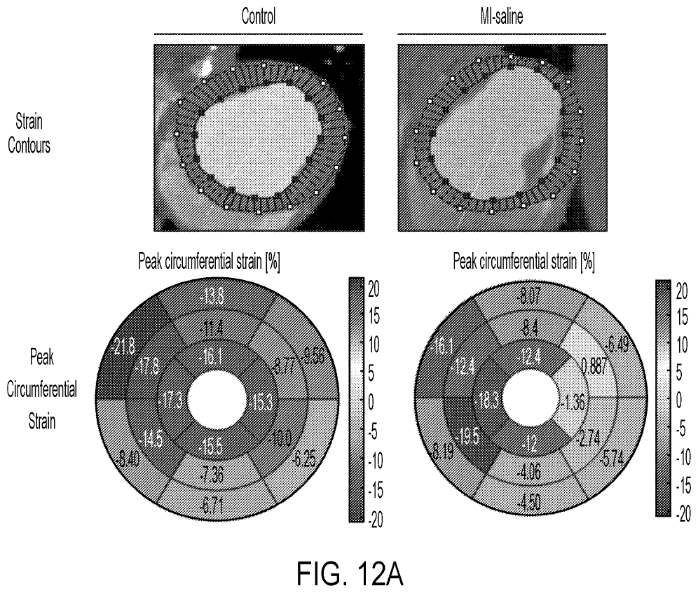

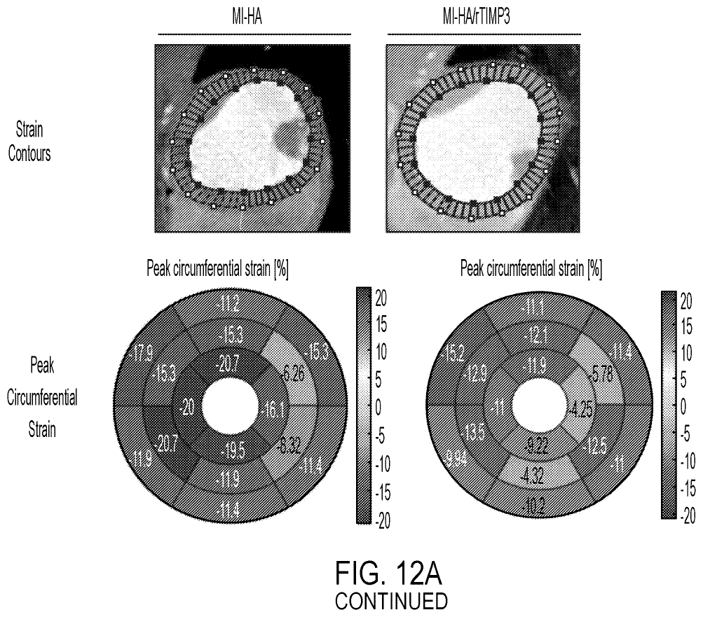

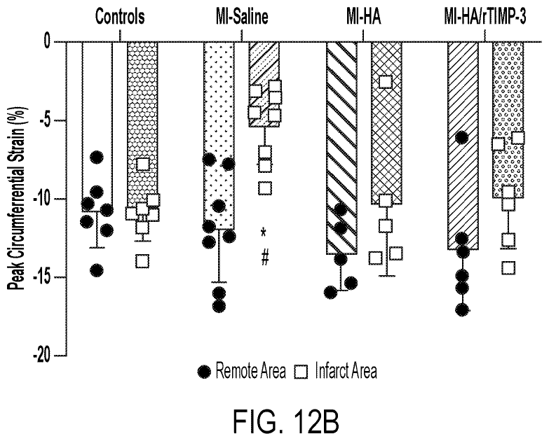

Representative short-axis CT images with superimposed endocardial and epicardial contours are shown for each group of pigs (FIG. 12A), along with corresponding color-coded polar maps of peak circumferential strain (FIG. 12A). Peak circumferential strain was significantly (p=0.008) reduced in the MI region of the MI-saline group compared with controls, while peak circumferential strain in the MI region was significantly greater than MI-Saline pigs in both MI-HA (p=0.039) and MI-HA/rTIMP-3 (p=0.049) groups (FIG. 12B). There was no decrease in peak circumferential strain in the infarct relative to remote area for either the MI-HA (p=0.279) or MI-HA/rTIMP-3 (p=0.085) groups, whereas the MI-Saline pigs had significantly reduced strain in MI area (p=0.006) (FIG. 12B).

### Example 6

Myocardial Perfusion and ^{99m}Tc-RP805 Uptake

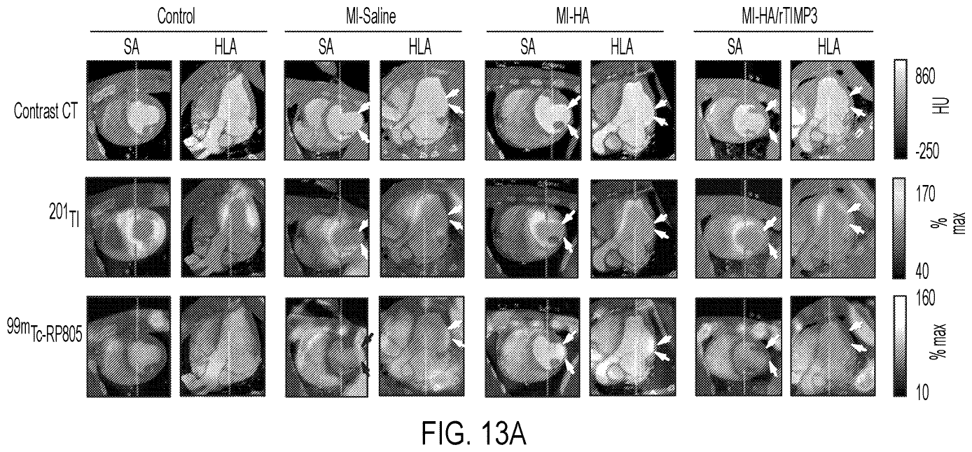

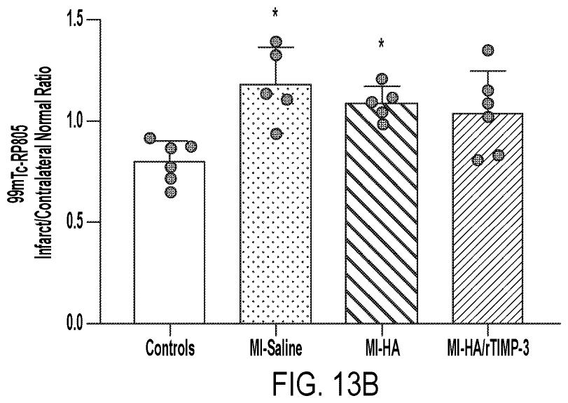

In Vivo Imaging: Hybrid ^{99m}Tc-RP805 and ²⁰¹T1 SPECT/CT imaging demonstrated focal uptake of ^{99m}Tc-RP805 in the ²⁰¹T1 perfusion defect in most pigs, although myocardial ^{99m}Tc-RP805 uptake of was greatest in the MI pigs that did not receive hydrogel (FIG. 13A). Quantification of in vivo ^{99m}Tc-RP805 analysis demonstrated a significant increase in the ratio of mid-inferolateral and mid-anterolateral regions to mid-inferoseptal and mid-anteroseptal regions in the MI-Saline (p=0.003) and MI-HA (p=0.027) groups compared to controls, but not in the MI-HA/rTIMP3 group (p=0.058) (FIG. 13B).

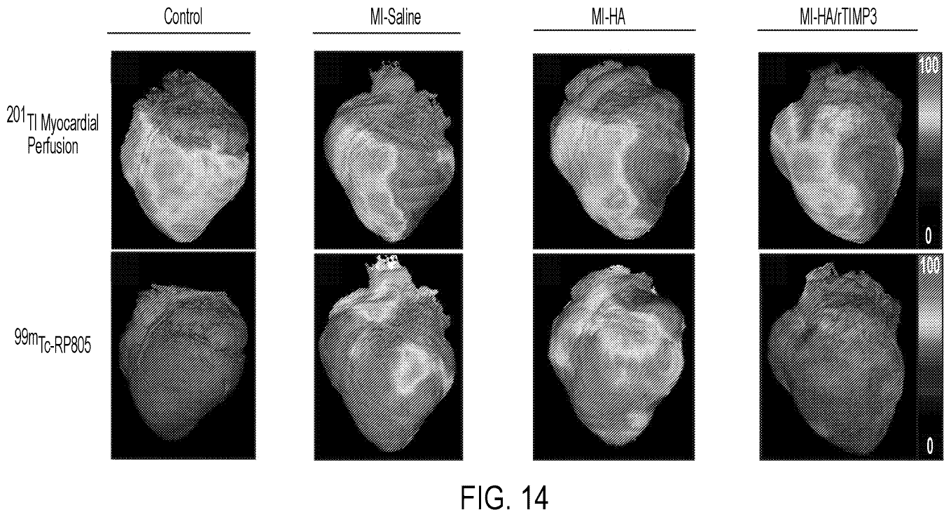

Ex-Vivo SPECT/CT Imaging: Representative ex vivo hybrid SPECT/CT images demonstrate the regional distribution of ^{99m}Tc-RP805 uptake and ²⁰¹T1 perfusion among each of the experimental groups (FIG. 14). The ²⁰¹T1 perfusion defects were similar between the MI groups. Control hearts demonstrated normal myocardial perfusion and no significant uptake of ^{99m}Tc-RP805 in the heart. ^{99m}Tc-RP805 uptake was seen within the MI area and left atrium in both MI-saline and MI-HA hearts. Heterogeneity of ^{99m}Tc-RP805 uptake in the infarct and peri-infarct area can be attributed to partial volume effects. The MI-HA/rTIMP-3 hearts demonstrated very little ^{99m}Tc-RP805 uptake in the LV although some activity was seen in the left atrium. Therefore, the local delivery of rTIMP-3 in the MI-HA/rTIMP-3 group almost completely suppressed ^{99m}Tc-RP805 uptake in the MI region.

### Example 7

### Gamma Well Counting:

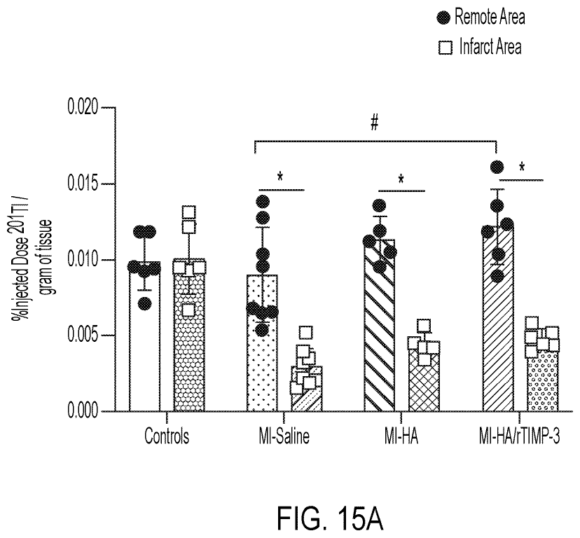

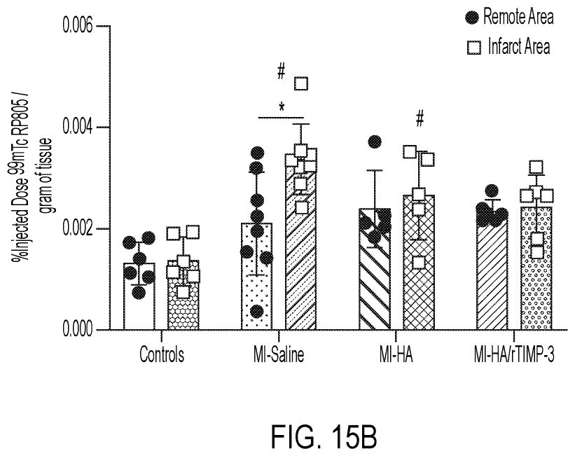

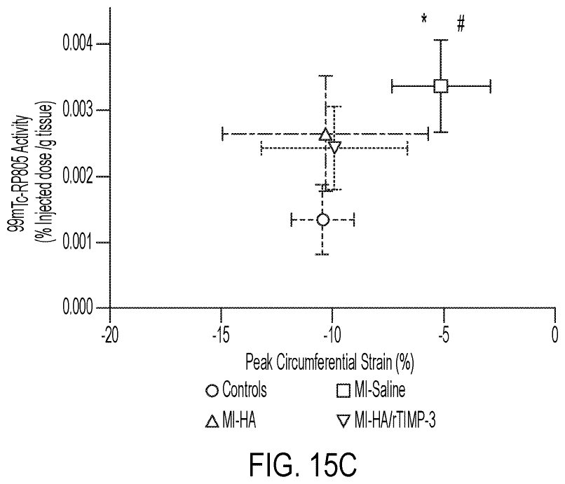

Left Ventricular Myocardial Activity: The imaging results were confirmed by tissue gamma well counting. ²⁰¹T1 uptake was uniform in the control pigs and significantly decreased in the infarct region in all MI groups (FIG. 15A).^{99m}Tc-RP805 uptake was significantly increased over 2-fold within the MI region in the MI-Saline group when compared to the remote region (p<0.001) and the uptake in control pigs (FIG. 15B). ^{99m}Tc-RP805 uptake in the MI region compared to the control pigs was only increased in the MI-Saline (p<0.001) and MI-HA group (p=0.020), while there was no significant increase in ^{99m}Tc-RP805 uptake in MI region of the MI-HA/rTIMP-3 group (p=0.051).

^{99m}Tc-RP805 Activity Versus Peak Circumferential Strain in MI Region: There was a significant increase in ^{99m}Tc-RP805 retention in the MI-Saline group in association with a significant decline in peak circumferential strain (FIG. 15C).

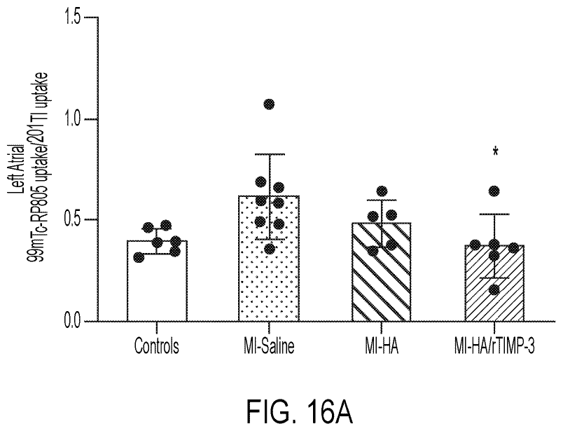

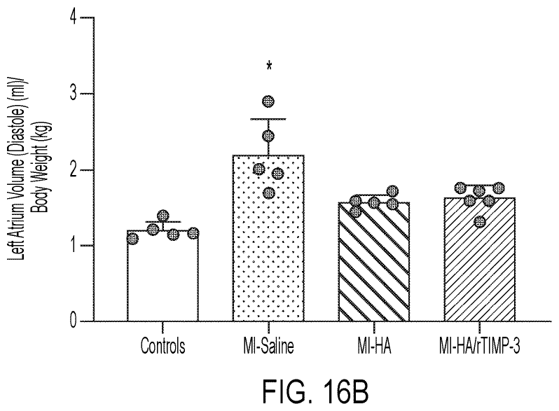

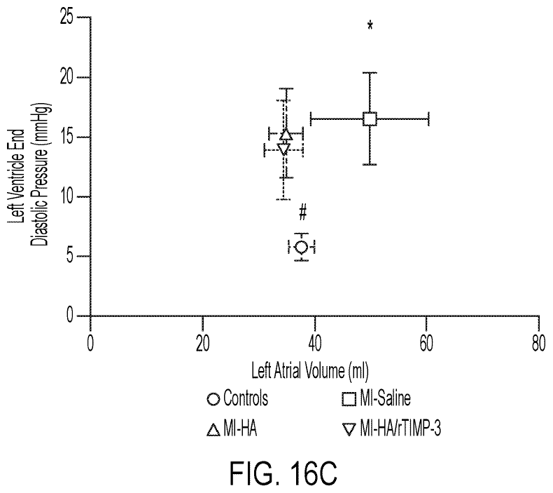

Left Atrial Myocardial Activity and Volumes: The ratio of ^{99m}Tc-RP805 uptake relative to ²⁰¹T1 uptake in the left atrium was significantly (p=0.041) reduced only in MI-HA/rTIMP-3 group compared to the MI-Saline group (FIG. 16A). This corresponded to a significant increase in left atrial diastolic volumes indexed to body weight in the MI-Saline group compared to controls (p<0.0001), with no significant difference found for either the MI-HA (p=0.153) or MI-HA/rTIMP3 groups (p=0.073) (FIG. 16B). At this early timepoint post-MI, this hydrogel effect on LA size was not associated with a decrease in LVEDP as all MI groups had an increase in LVEDP compared to controls (p<0.01) (FIG. 16C). Together, these results suggest that local intramyocardial delivery of hydrogel to the MI region, and specifically the local delivery of r-TIMP3 may also prevented post-MI atrial remodeling.

### Example 8

### Ex Vivo Infarct Size

Infarct size as a percentage of LV was 9±3% in MI-Saline group, significant reduced in in the MI-HA/rTIMP-3 group (5±2%, p=0.035), but not in MI-HA group (6±2%, p=0.118).

### DISCUSSION

The present study has demonstrated that intramyocardial delivery of a HA hydrogel in the infarct region post-MI with or without local delivery of rTIMP-3 can increase wall thickness in infarct region, reducing wall stress, and LV end-diastolic volume while preserving circumferential strain though a reduction in myocardial MMP activation within the MI region in a clinically relevant porcine model of post-MI remodeling. The suppression of MMP activation as assessed by myocardial ^{99m}Tc-RP805 uptake was associated with an improvement in regional function. The observed changes in regional MMP activation and myocardial perfusion were assessed noninvasively with hybrid SPECT/CT imaging. The local delivery and sustained release of rTIMP-3 directly into the MI region provided the added benefit of reducing infarct size. The use of contrast cine CT imaging in conjunction with dual isotope ^{99m}Tc-RP805/²01T1 SPECT/CT imaging was able to relate the changes in regional myocardial perfusion and MMP activation to changes in regional myocardial thickness and strain following the intramyocardial delivery of therapeutic hydrogels.

Significant wall thinning in the MI region of the MI-saline group was observed, while both HA hydrogel groups demonstrated a significant an improvement in circumferential strain and an increase in myocardial wall thickness of the infarct area, which reduce regional wall stress based on LaPlace's law. Recent studies have indicated that strain assessed with echocardiography can have predictive value on myocardial functional recovery. The use of hybrid SPECT/CT imaging and low dose iodinated contrast that delineated endocardial and epicardial surfaces of the LV allowed for assessment of not only regional myocardial circumferential strain and wall stress, but could facilitate partial volume correction of radiotracer uptake, providing a more accurate estimate of both perfusion and MMP activity in spite of the changes in wall thickness and function.

Sustained release of rTIMP-3 from a hydrogel provided inhibition of tissue MMP-2, MMP-9, and MMP-14 activity, along with a reduction in multiple markers of inflammation (MCP1, TNF, IL-8, IL-10) and collagen type 1 and 3 that was not seen with a hydrogel that released a non-functional truncated TIMP-3 was observed previously. According to the present disclosure, some increase in ^{99m}Tc-RP805 uptake was observed in the infarct region over control pigs injected with hydrogel alone, which was not seen with the rTIMP-3 releasing hydrogel. a direct correlation between tissues MMP proteolytic activity determined by zymography and expression levels assessed by RT-PCR compared with ^{99m}Tc-RP805 uptake has been previously demonstrated, including a study using the same porcine MI model of permanent coronary occlusion. Thus, this multimodality imaging approach offers a noninvasive method to potentially guide and evaluate myocardial delivery of hydrogels along with delivery of molecularly targeted therapeutics. The current evaluation of this type of therapy is often relegated to evaluation of only functional or clinical indices, without demonstration of the underlying biological effect. The present disclosure contemplates the ability of hybrid SPECT/CT imaging to track differences in MMP activation in conjunction with structural changes post-MI.

Previous basic and clinical studies have identified that the induction of MMPs, and reduction in endogenous TIMPs play a contributory role in early ischemic injury and late LV remodeling. Circulating MMP plasma levels are elevated in patients following MI and appears to serve as a predictor for development of heart failure. However, despite a large number of mechanistic studies implicating MMP activation and adverse LV remodeling, clinical trials employing systemic pharmacological MMP inhibition have yielded mixed results (TIPTOP, PREMIER). This may be due in part, to the lack of a direct approach to assess MMP activation within the LV myocardium in vivo. Previous studies have investigated the use of targeted TIMP-3 delivery to the MI region and reported reduced adverse post-MI remodeling. More recently, it was established that intracoronary delivery of rTIMP-3 in pigs at the time of reperfusion favorably reduced MMP activity and LV remodeling and indices of heart failure progression. The present disclosure critically advances this field of injectable therapeutic hydrogels by providing a means by which to monitor the effects of local hydrogel delivery and co-register MMP-targeted imaging with regional and global indices of perfusion and function. Specifically, MMP targeted SPECT radiotracer, ^{99m}Tc-RP805, was evaluated. Previous work demonstrated that ^{99m}Tc-RP805 uptake was increased within the MI region at one week post MI induced by permanent coronary occlusion, and this increase was correlated to changes in the activity of several specific MMP subtypes known to play are role in post-MI remodeling.

Interestingly, the present inventors observed increased ^{99m}Tc-RP805 uptake in the left atrium associated with an increase in LA volume post-MI in the MI-saline group presumably due to both pressure and volume overload associated with mitral regurgitation identified by Doppler echocardiography performed in only a subset of the pigs, and a significant reduction in relative ^{99m}Tc-RP805 uptake in the left atrium without an increase in LA size in the MI-HA/rTIMP-3 pigs when compared to untreated MI-Saline pigs. Left atrial remodeling is a known phenomenon following acute MI, and an important determinant of subsequent morbidity and mortality. While these data are preliminary, it does suggest potential additional beneficial effects of the hydrogel delivery of rTIMP-3 in reducing LV remodeling and associated effect on MMP activation in the atria and atrial LA remodeling.

A surgical model of permanent coronary occlusion was employed, which was associated with significant MMP activation in the chest wall immediately adjacent to the infarct complicating in vivo imaging. Studies are underway to analyze hydrogels delivered at variable timepoints following ischemia-reperfusion injury induced by 90 min percutaneous coronary balloon occlusion. These studies are focused on serial dynamic in vivo ^{99m}Tc-RP805 hybrid SPECT/CT imaging for defining the optimal timing of imaging and a quantitative approach for guiding local delivery of therapeutic hydrogels to the heart.

In certain embodiments, studies were carried out with a ^{99m}Tc-labeled MMP-targeted SPECT imaging agent in combination with ²⁰¹T1 imaging. There are other MMP-targeted PET imaging agents, such as ¹⁸F-IPFP, that might offer other advantages due to the improved image resolution and sensitivity provided by PET imaging. The current studies were performed on a hybrid CZT SPECT 64-slice CT scanner that provides a 5-fold increased sensitivity over conventional sodium-iodide SPECT cameras along with a 2-fold improvement in resolution. The quantification of any targeted cardiac hot spot imaging agent benefits from a co-registered reference perfusion agent which is more easily accomplished with dual isotope SPECT imaging.

In conclusion, this study establishes the utility of a clinically relevant noninvasive imaging approach that enables direct visualization and quantification of regional myocardial MMP activation in relation to changes in critical physiological indices of regional perfusion and function. The application of this dual isotope hybrid SPECT/CT imaging approach may facilitate the optimization of hydrogel injections coupled to small molecular therapeutics, for prevention of early post-MI infarct expansion and subsequent late atrial and ventricular remodeling.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A method of imaging a subject's lung, the method comprising:
administering to the subject's lung an imaging agent comprising a ^{99m}Tc-labeled matrix metalloproteinase targeted radiotracer;
acquiring an image of the subject's lung using single-photon emission computed tomography/computed tomographic (SPECT/CT) imaging, optionally using dynamic imaging; and
measuring an amount of the imaging agent bound to matrix metalloproteinase in the subject's lung.

2. The method of claim 1, wherein the imaging agent is a ^{99m}Tc-labeled matrix metalloproteinase inhibitor.

3. The method of claim 1, wherein the imaging agent comprises ^{99m}Tc-RP805, ^{99m}Tc-pyrophosphate, or ^{99m}Tc-Maraciclatide.

4. The method of claim 1, wherein the subject is suspected of being afflicted with a pulmonary infection.

5. The method of claim 1, wherein the subject is suspected of being afflicted with acute respiratory distress syndrome (ARDS), coronavirus disease 2019 (COVID-19), chronic obstructive pulmonary disease (COPD), lymphangioleiomyomatosis (LAM), idiopathic pulmonary fibrosis (IPF), or acute lung injury (ALI).

6. The method of claim 1, wherein the subject is suspected of being afflicted with a vascular disease.

7. The method of claim 6, wherein the vascular disease comprises an ischemic stroke, hemorrhagic stroke, transient ischemic attack (TIA), vascular inflammation due to meningitis, atherosclerosis, arteritis, physical obstruction of arterial blood supply to the brain, lacunar stroke, hypoperfusion embodying diffuse injury, myocardial infarction and arrhythmia, restenosis associated with percutaneous transluminal coronary angioplasty, peripheral vascular disease and cerebral vascular disease, venous occlusive disorders such as deep vein thrombosis, hypercoagulopathies, aneurysms or chronic progressive vascular disease.

8. The method of claim 1, wherein the subject's lung is imaged at about 60 minutes to about 240 minutes after administration of the imaging agent.

9. The method of claim 1, wherein the subject's lung is imaged at about 90 minutes to about 180 minutes after administration of the imaging agent.

10. The method of claim 1, wherein the presence of the imaging agent bound to matrix metalloproteinase in an amount higher than an amount in a reference subject indicates the subject is afflicted with a viral or bacterial infection.

11. The method of claim 10, wherein the reference subject is a healthy subject.

12. The method of claim 1, wherein the matrix metalloproteinase (MMP) is selected from one or more of MMP-1, MMP-2, MMP-9, MMP-12, or MMP-14.

13. The method of claim 1, wherein the lung image is a left lung image.

14. The method of claim 1, wherein the lung image is a right lung image.

15. A method of evaluating a pulmonary infection in a subject, the method comprising:
administering an imaging agent comprising a ^{99m}Tc-labeled matrix metalloproteinase targeted radiotracer to a subject's lung;
acquiring an image of the subject's lung using single-photon emission computed tomography/computed tomographic (SPECT/CT) imaging, optionally using dynamic imaging; and
measuring an amount of the imaging agent bound to matrix metalloproteinase in the subject's lung,
wherein the presence of the imaging agent bound to a matrix metalloproteinase in an amount higher than an amount in a reference subject indicates the subject is afflicted with a pulmonary infection.

16. The method of claim 15, wherein the imaging agent is a ^{99m}Tc-labeled matrix metalloproteinase inhibitor.

17. The method of claim 15, wherein the imaging agent comprises ^{99m}Tc-RP805, ^{99m}Tc-pyrophosphate, or ^{99m}Tc-Maraciclatide.

18. The method of claim 15, wherein the subject is suspected of being afflicted with acute respiratory distress syndrome (ARDS), coronavirus disease 2019 (COVID-19), chronic obstructive pulmonary disease (COPD), lymphangioleiomyomatosis (LAM), idiopathic pulmonary fibrosis (IPF), or acute lung injury (ALI).

19. The method of claim 15, wherein the subject's lung is imaged at about 60 minutes to about 240 minutes after administration of the imaging agent.

20. The method of claim 15, wherein the subject's lung is imaged at about 90 minutes to about 180 minutes after administration of the imaging agent.

21. The method of claim 15, wherein the reference subject is a healthy subject.

22. The method of claim 15, wherein the matrix metalloproteinase (MMP) is selected from one or more of MMP-1, MMP-9, MMP-12, or MMP-13.

23. The method of claim 15, wherein the lung image is a left lung image.

24. The method of claim 15, wherein the lung image is a right lung image.

25. The method of claim 15, wherein the imaging agent is present from about 20% to about 75% more in a subject's lung compared to a reference subject.

26. A method of high resolution imaging of a subject's heart tissue, the method comprising:
administering to the subject's heart tissue a first imaging agent comprising a ^{99m}Tc-labeled matrix metalloproteinase targeted radiotracer;
acquiring an image of the subject's heart tissue using single-photon emission computed tomography/computed tomographic (SPECT/CT) imaging, optionally using dynamic imaging; and
quantifying an amount of the first imaging agent bound to a matrix metalloproteinase in the subject's heart tissue.

27. The method of claim 26, wherein the first imaging agent is a ^{99m}Tc-labeled matrix metalloproteinase inhibitor.

28. The method of claim 26, wherein the first imaging agent comprises ^{99m}Tc-RP805, ^{99m}Tc-pyrophosphate, or ^{99m}Tc-Maraciclatide.

29. The method of claim 26, further comprising administering a second imaging agent at from about 200 minutes to about 300 minutes after administration of the first imaging agent.

30. The method of claim 29, wherein the second imaging agent comprises ²⁰¹Thallium.

31. The method of claim 26, wherein the subject is suspected of being afflicted with a vascular disease.

32. The method of claim 26, wherein the vascular disease comprises an ischemic stroke, hemorrhagic stroke, transient ischemic attack (TIA), vascular inflammation due to meningitis, atherosclerosis, arteritis, physical obstruction of arterial blood supply to the brain, lacunar stroke, hypoperfusion embodying diffuse injury, myocardial infarction and arrhythmia, restenosis associated with percutaneous transluminal coronary angioplasty, peripheral vascular disease and cerebral vascular disease, venous occlusive disorders such as deep vein thrombosis, hypercoagulopathies, aneurysms or chronic progressive vascular disease.

33. The method of claim 26, wherein the subject's heart tissue is imaged at about 60 minutes to about 240 minutes after administration of the first imaging agent.

34. The method of claim 26, wherein the subject's heart tissue is imaged at about 90 minutes to about 180 minutes after administration of the first imaging agent.

35. The method of claim 26, wherein the reference subject is a healthy subject.

36. The method of claim 26, wherein the matrix metalloproteinase (MMP) is selected from one or more of MMP-1, MMP-9, MMP-12, or MMP-13.

37. The method of claim 26, wherein the heart tissue comprises a left atrium.

38. The method of claim 26, wherein the imaging agent is present from about 20% to about 75% more in a subject's lung compared to a reference subject.
